(19) United States
(12) Patent Application Publication
(10) Pub. No.: US 2004/0265880 A1
(43) Pub. Date: Dec. 30, 2004

(54) Genomic and proteomic approaches for the development of cell culture medium

(75) Inventors: Laurel M. Donahue, St. Louis, MO (US); Daniel W. Allison, St. Louis, MO (US); Terrell K. Johnson, St. Louis, MO (US); Kathryn A. Aboytes, St. Louis, MO (US); Danny Fong, St. Louis, MO (US)

Correspondence Address: SENNIGER POWERS LEAVITT AND ROEDEL, ONE METROPOLITAN SQUARE, 16TH FLOOR, ST LOUIS, MO, 63102, US

(73) Assignee: Sigma-Aldrich Co.

(21) Appl. No.: 10/842,649

(22) Filed: May 10, 2004

Related U.S. Application Data
(60) Provisional application No. 60/469,578, filed on May 9, 2003.

(51) Int. Cl. C12Q 1/68; C12N 5/00

(52) U.S. Cl. 435/6; 435/404

(57) ABSTRACT

The present invention relates to a method for designing a cell culture medium adapted to support a cell line in a pre-defined manner. The method includes generating an expression profile of a cell line, identifying from the expression profile a set of biomolecules to evaluate for their effect on an end-point assay using the cell line, testing each biomolecule in the set for its effect in the endpoint assay, wherein each biomolecule that is determined to have a measurable effect in the end-point assay relative to a control, not containing the biomolecule, is considered a positive biomolecule and formulating a cell culture medium for the cell line by adding a positive biomolecule to a basal medium to form a modified medium, and determining whether the modified medium is sufficient to support the cell line in the pre-defined manner. Methods for identifying biomolecules for use in designing a cell culture medium adapted to support a cell line in a pre-defined manner, methods for preparing a serum-free cell culture medium that is sufficient to support a cell line and arrays for use in such methods are also provided.

### CROSS-REFERENCE TO RELATED APPLICATIONS

This application is a nonprovisional of U.S. patent application Ser. No. 60/469,578, filed May 9, 2003. The entire text of that application is hereby incorporated herein by reference.

### FIELD OF THE INVENTION

The present invention relates to methods and materials for designing and optimizing cell culture medium. More particularly, the present invention relates to methods and materials for making cell culture medium that are adapted to support cell lines in pre-defined manners.

### BACKGROUND OF THE INVENTION

Tissue was first cultured in the early 1900s and was largely derived from lower vertebrates. Scattered fragments of tissue were kept alive in dishes and cells migrated from the explant. Occasionally, cells divided. In the 1940s and 1950s, it became possible to take explants of avian or mammalian tissue and derive either normal cells, or in the case of rodents, continuous cell lines. These cells were first cultured in a basal medium, which included some building blocks of the cell's components, such as amino acids, vitamins, salts, etc. In addition, the basal medium was often supplemented with embryo extracts or mammalian serum. Embryo extracts and serum contain thousands of components, including growth factors, cytokines, hormones, attachment factors and other unknown components that promote cell survival and proliferation in vitro. Cells, generally, will not proliferate when cultured only in basal medium, which has not been supplemented with serum.

In the 1970s, investigators started formulating medium that were defined, extract-free and serum-free. See e.g., L. Defrancesco (1998) Serum-Free Cell Culture: From Art to Science, The Scientist 12[1]:19; R. G. Ham and W. L. McKeehan (1979) Media and Growth Requirements, Methods in Enzymology, vol LV111:44; and J. Bottenstein et al. (1979) The Growth of Cells in Serum-free Hormone-Supplemented Media, Methods in Enzymology, vol LV111:94. This endeavor continues to this day. Because of the complexity of serum, however, it has been challenging to identify the components of serum that provide cell type-specific growth.

Concurrent with the effort to develop serum-free medium has been a continuous study of cell nutritional biochemistry. This has allowed for improved basal formulation (carbon source, amino acids, vitamins, trace metals, etc.).

Medium is currently formulated based on prior knowledge of cell nutritional biochemistry and the knowledge contained in previously published sources to identify additional components, such as growth factors, that have been shown to have a positive, proliferative effect on a particular cell line. In addition, as resources permit, an investigator may undertake some amount of random screening of factors that may have a positive effect (for example, growth factors described in the literature for an unrelated cell type). The use of random screening has benefited from the miniaturization of assay formats, as well as the use of statistical approaches to experimental design that allow for fewer test conditions to be examined. See e.g., S. Peppers et al. (2001) Performance-Optimized Hybridoma Medium: Replacing Serum and Other Animal-Derived Components, Life Science Quarterly, Sigma-Aldrich Technical Application Newsletter, volume 2[2]; C.-H. Liu et al. (2001) Factorial Designs Combined with the Steepest Ascent Method to Optimize Serum-Free Media for CHO Cells, Enzyme and Microbial Technology 28:314; and E. J. Kim et al. (1998) Development of a Serum-Free Medium for the Production of Humanized Antibody from Chinese Hamster Ovary Cells using a Statistical Design, In Vitro Cell and Developmental Biology, 34:757.

The requirement for serum by most cell lines is also a complicating factor for cells that are used in the production of, e.g., human biologics. For example, certain hybridoma cell lines, which are used to make therapeutic antibodies, may require serum in the cell culture medium for proper growth and proliferation. Before the antibodies generated by such hybridomas are used in a human patient, it is desirable to remove all serum components, which might cause disease, e.g., prions that cause spongiform encephalitis in humans. Such purification methods are cumbersome, expensive and not always completely reliable.

Accordingly, it is advantageous to optimize, e.g. hybridoma medium, to support the growth and proliferation of a particular hybridoma cell line in a low or preferably serum-free medium. Heretofore, such optimization required a trial and error approach to identifying components for a cell culture medium with no regard to the specific requirements of the cell line. At best, the investigator relied on his or her own previous experience or what could be learned from the scientific literature. Such a procedure would benefit from a more directed approach based on known requirements by the cell.

The field of molecular biology, in particular genomics and proteomics, offers efficient methods for identifying, in a single experiment, large numbers of genes or proteins that are transcribed or expressed by a cell. For example, microarray analysis is a technique for quickly and efficiently identifying expression patterns of hundreds of expressed genes in a single test.

Microarray analysis has been used, for example, to show differential gene expression of a cell type or tissue cultured under different conditions, or a cell type from a normal individual or tissue compared to that same cell type or tissue from an individual with a specific disease or condition. See lyer et al (1999) The Transcriptional Program in the Response of Human Fibroblasts to Serum, Science 283:83. In addition, one group has used genomic and proteomic approaches to examine changes in gene expression upon shifting metabolic states of a particular cell. See Korke et al (2002) Genomic and Proteomic Perspectives in Cell Culture Engineering, Journal of Biotechnology 94:73. Another group reported using proteomics and gene arrays, in combination with metabolite data, to identify changes over the course of culture and between cells lines, comparing the glycosylation of one CHO cell line to the glycosylation of another CHO line. See, Andersen, Engineering Conference International, Cell Culture Engineering IX, Session 4, Cell Engineering (Abstract) (2004). Microarray analysis, however, has not been applied to developing and/or optimizing cell culture medium for specific cell lines.

### SUMMARY OF THE INVENTION

Among the various aspects of the present invention is a rational method of formulating a cell culture medium having a desired effect upon cell growth, cell proliferation or even protein expression. Advantageously, random trial and error approaches to the formulation of a cell culture medium need not be employed.

Briefly, therefore, the present invention is directed to a method of formulating a cell culture medium, the method comprising detecting a nucleic acid or an expressed amino acid sequence in a cell. Using information derived from this detection, a cell culture medium is formulated to contain a molecule which modulates a cellular process in a desired manner.

The present invention is further directed to a method of preparing a cell culture medium in which an array of immobilized biopolymers are contacted with a sequence derived from a cell. The sequence may be a polynucleotide or its complement derived from a cell. Alternatively, the sequence may be a polypeptide derived from a cell. If binding is detected, a molecule is selected for inclusion in a cell culture medium and tested for its effect based upon a cellular process which is, in some manner, revealed or affected by the polynucleotide or polypeptide.

One embodiment of the invention is a method for designing a cell culture medium adapted to support a cell line in a pre-defined manner. This method comprises generating an expression profile of a cell line; identifying from the expression profile a set of biomolecules to evaluate for their effect on an endpoint assay using the cell line; testing each biomolecule in the set for its effect in the endpoint assay, wherein each biomolecule that is determined to have a measurable effect in the endpoint assay relative to a control, not containing the biomolecule, is considered a positive biomolecule; and formulating a cell culture medium for the cell line by adding a positive biomolecule to a basal medium to form a modified medium, and determining whether the modified medium is sufficient to support the cell line in the pre-defined manner.

Another embodiment is a method for identifying biomolecules for use in designing a cell culture medium adapted to support a cell line in a pre-defined manner. This method comprises generating a pool of polynucleotide probes that are complementary to polynucleotide sequences that encode fragments of polypeptides expressed by a cell line; contacting the pool of polynucleotide probes under hybridizing conditions with at least one array comprising a plurality of biopolymers immobilized on the surface of the array, each biopolymer encoding a fragment of a distinct polypeptide that participates in a biologically significant cellular process; generating an expression profile by detecting each polynucleotide probe that hybridizes to each biopolymer; and selecting biomolecules, based on the expression profile, to be candidate components for a cell culture medium based on the function of the polypeptide partially encoded by the biopolymers to which a probe hybridizes.

A further embodiment of the invention is a method for preparing a serum-free cell culture medium that is sufficient to support a cell line. This method comprises generating a pool of polynucleotide probes that are complementary to polynucleotide sequences that encode fragments of polypeptides expressed by a cell line; contacting the pool of polynucleotide probes under hybridizing conditions with at least one array comprising a plurality of biopolymers immobilized on the surface of the array, each biopolymer encoding a fragment of a distinct polypeptide that participates in a biologically significant cellular process; generating an expression profile by detecting each polynucleotide probe that hybridizes to each biopolymer; selecting biomolecules, based on the expression profile, to be candidate components for a serum-free medium based on the function of the polypeptide partially encoded by the biopolymers to which a probe hybridizes; testing each candidate component to determine its effect on growth and/or proliferation of the cell line, and designating those candidate components that increase growth and/or proliferation of the cell line as positive biomolecules; and adding a positive biomolecule to a serum-free basal medium and evaluating the growth and/or proliferation of the cell line in the modified medium.

A still further embodiment of the invention is a cell culture medium made by any of the processes set forth above.

Another embodiment of the invention is an array for designing and/or optimizing cell culture medium. The array comprises a pool of biopolymers immobilized on a surface of a substrate, each biopolymer selected from the group consisting of polynucleotides encoding fragments of distinct polypeptides that are participants in biologically significant cellular processes, antibodies or antibody fragments that specifically bind to polypeptides that are participants in biologically significant cellular processes and fragments of polypeptides that are participants in biologically significant cellular processes.

### BRIEF DESCRIPTION OF THE FIGURES

FIG. 1 shows a profile of HEK-293 cells on a microarray.

FIGS. 2A-2D show graphs of RFU values for four (4) growth factors identified from Table 2 that exhibited “positive effects” in a HEK-293 proliferation assay.

FIGS. 3A-3D show graphs of RFU values for four (4) growth factors identified from Table 2 that exhibited “no effect” in a HEK-293 proliferation assay.

FIG. 4 shows pictures of HEK-293 grown on (A) untreated substrate; (B) collagen I coated substrate; and (C) collagen IV coated substrate.

FIGS. 5A-5D graphically depict RFU values for basic fibroblast growth factor (5A), platelet-derived growth factor AB (5B), stromal cell-derived factor 1 a (5C), and interleukin-1 (5D) identified from Table 4 that exhibited “positive effects” in a proliferation assay using WI-38 cells.

FIGS. 6A-6C graphically depict the additive effects of growth factors by illustrating the RFU values for 1% FBS with bFGF and PDGF AB (6A), 0.5% FBS with bFGF and PDGF AB (6B), and 0% FBS with bFGF and PDGF (6C) identified from studies of Example 1.

FIGS. 7A-7C graphically depict the results of the study carried out in Example 3, wherein PCR techniques were used to identify beta-actin, CCR7, and PDGFRA sequences.

FIGS. 8A and 8B graphically depict the results of the studies carried out in Example 2, wherein a chemiluminescent macroarray (8A) and a fluorescent antibody array (8B) for WI-38 were produced.

FIGS. 9A and 9B graphically depict the results of the study carried out in Example 1, illustrating the positive effects of interleukin-1 on proliferation of CHO-AP (9A) and the positive effects of interleukin-1 on productivity (alkaline phosphatase production) of CHO-AP (9B).

FIGS. 10A and 10B graphically depict the results of the studies carried out in Example 4, illustrating the endogenous intermediates which exhibited positive effects on the proliferation of HEK-293 cells.

### DETAILED DESCRIPTION OF THE INVENTION

The present invention generally relates to the use of sequences (nucleic acid or expressed amino acid) present in a cell to formulate a cell culture medium specific for the support, growth, proliferation, division, metabolism, or adhesion of a cell. Examples of nucleic acid sequences that may be detected include, for example, DNA and RNA sequences and mutant DNA and RNA sequences. In one embodiment, the nucleic acid sequence that is detected is an mRNA sequence. The nucleic acid sequence may code for a polypeptide or a fragment of a polypeptide, or may be a non-coding region, such as, for example an intron or a regulatory sequence. In another embodiment of the invention, the nucleic acid sequence is a nucleic acid analog, such as for example a peptide nucleic acid (PNA). Examples of expressed amino acid sequences include, for example, proteins, fragments of proteins, and polypeptides. In one embodiment, the expressed amino acid sequence that is detected is a polypeptide that encodes all or a portion of a protein expressed in the cell.

Generally, the cell culture medium is formulated according to a method comprising a multi-step process comprising a step of detecting a sequence in a cell line and a step of formulating a cell culture medium to contain a molecule to modulate the detected sequence or its expression or to modulate a cellular process affected by the detected sequence or its expression. In one preferred embodiment, the method further comprises an intervening step of determining whether the molecule selected for the formulation modulates the sequence or its expression or modulates a cellular process affected by the sequence or its expression.

The sequence may be detected using methods conventionally used for detecting a specific nucleic acid sequence or an expressed amino acid sequence. Techniques for detecting a nucleic acid sequence include, for example, the use of genomic methods such as the screening of arrays (both micro- and macroarrays), as described, for example in Duggan et al., Nature Genetics Supplement, 21: 10-14 (1999) (microarrays) and in Example 1 of the present application; PCR based techniques as described, for example, in Example 3 (standard reverse transcription (RT) PCR and real-time quantitative RT-PCR) of the present application; and antibody arrays. Techniques for detecting an expressed amino acid sequence include, for example, the use of proteomic methods such as the screening of arrays (both micro- and macroarrays) as described, for example in, MacBeath et al., Science, 289:1760-1763 (2000); Ge, H. UPA, Nucleic Acids Res 28:e3 (2000); Lueking. et al. Anal Biochem 270:103-111 (1999); Arenkov et al., Anal Biochem 278:123-131 (2000); and Sreekumar et al., Cancer Research 61:7585-7593 (2001) (microarrays) and in Example 2 of the present application; antibody arrays (Haab et al., Genome Biology, 2(2): Research 0004.1-0004.13 (2001); Sreekumaret al., Cancer Research 61:7585-7593 (2001); and Example 2 of the present specification); and western blotting (Harlow et al., Antibodies: A Laboratory Manual, Cold Spring Harbor Laboratory Press, Plainview, N.Y. (1988); Bjerrum et al., N.H.H. CRC Handbook of Immunoblotting of Proteins, Volume I, Technical Descriptions, CRC Press, (1988) p. 229-236; and Dunbar, (ed.) Protein Blotting: A Practical Approach, IRL Press, NY, p. 67-70 (1994))

In a preferred embodiment, the method further comprises an intermediate step between the detecting and the formulating steps of determining whether a molecule modulates the detected sequence or its expression or modulates a cellular process affected by the detected sequence or its expression. Such a determination may be made, for example, by observing changes in cellular activities involved in the support, the growth, the proliferation, the metabolism, the control of the cell cycle and the division of a cell. Observations of the changes may be made by conventional tests or assays, such as those described below with respect to end-point assays. These assays include, for example, proliferation assays (such as, for example, manual counting of cells, DNA content assays, protein content assays, and metabolic assays (such as, for example, the resazurin assay described below in Example 5)), adhesion assays (such as, for example, plating efficiency assays, focal adhesion assays, and the adhesion assay described below in Example 5), production assays (such as, for example, the alkaline phosphatase assay described in Example 5), cell metabolism assays (such as, for example, assays which monitor the use of a particular substance or the production of a particular by-product), differentiation assays (such as, for example, morphological assays and assays which demonstrate changes in gene expression or function of a cell), and apoptosis assays. The results obtained from these assays may then be used to select a molecule to affect a desired cellular activity in a desired manner. Advantageously, the determination step is not required, although it may be preferred in some instances. Likewise, this step may also be omitted under other instances.

By way of example, increased cell proliferation may be controlled by a particular molecule, such as, for example, the growth factor PDGF. A determination of whether PDGF affects a cellular activity involved with proliferation in a desired manner may be made using a proliferation assay, such as, for example, a DNA content assay or by manual counting of cells. Likewise, increased cell adhesion may be controlled by a particular molecule, such as, for example, a particular integrin. A determination of whether a particular integrin affects a cellular activity involved with cell adhesion in a desired manner may be made using an adhesion assay, such as, for example, a plating efficiency assay.

The formulation of the cell culture medium may be achieved by the addition of a molecule to modulate a cellular activity to a culture medium. This may be accomplished by simply adding the molecule to a known medium or creating a medium containing the molecule. In either instance, the molecule may be added in an amount sufficient to modulate the detected sequence or its expression or to modulate a cellular process affected by the detected sequence or its expression. Such a modulation may be, for example, an increase in the detected sequence or its expression or a cellular process affected by the detected sequence or its expression, the decrease in the same, or an increase of one with respect to a particular detected sequence or its expression or a cellular process affected by the detected sequence or its expression and a decrease with respect to a different detected sequence or its expression or a cellular process affected by the detected sequence or its expression.

By way of example, if the cell culture medium is formulated according to the present methods for the growth or proliferation of Chinese hamster ovary (CHO) cells, a nucleic acid or expressed amino acid sequence may be detected by using a microarray generally containing biopolymers such as, for example, Chinese hamster cell receptors, and CHO receptors in particular, and including, for example, the IGF-1 receptor, the bFGF receptor, and estrogen receptors, that would hybridize or bind to sequences related to CHO cell growth or proliferation. A determination of whether a molecule modulates a cellular activity related to growth or proliferation of CHO cells may be achieved by using, for example, manual counting of cells, DNA content assays, protein content assays, metabolic assays (such as, for example, the resazurin assay described below in Example 5), and production assays (such as, for example, the alkaline phosphatase assay described in Example 5). The cell medium would then be formulated to contain a molecule using the information obtained from these steps.

In still a further example, if the cell culture medium is formulated according to the present methods for the support or maintenance of Chinese hamster ovary (CHO) cells, a nucleic acid or expressed amino acid sequence may be detected by using a microarray generally containing biopolymers such as, for example, those disclosed above with respect to cell growth and proliferation, as well as metabolic enzymes, including, for example, enzymes involved in glycolysis, the TCA cycle, protein glycosylation, and protein targeting and secretion, that would hybridize or bind to sequences related to CHO cell support or maintenance. A determination of whether a molecule modulates a cellular activity involved in the support or maintenance of CHO cells may be achieved by using, for example, manual counting of cells, DNA content assays, protein content assays, and metabolic assays (such as, for example, the resazurin assay described below in Example 5). The cell medium would then be formulated to contain a molecule using the information obtained from these steps.

The methods described herein may be used to formulate a cell culture medium for a range of cell types. For example, the cells may be germ cells or somatic cells. The cells may be animal cells, including cells from vertebrates and invertebrates, insect cells, bacterial cells, plant cells, or fungal cells. The cells may be derived from a single cell type or may be derived from multiple cell types, such as, for example, in a multicellular tissue or organ. Moreover, the cell type and source used to formulate the cell culture medium may be different from the cell type and source of the cell subsequently supported, grown, or proliferated in the formulated medium.

In one preferred embodiment, the cell is from or part of a particular cell line. As used herein “cell line” means a cell from a given source, e.g., a tissue, or organ, or a cell in a given state of differentiation, or a cell associated with a given pathology or genetic makeup. “Cell line” encompasses cells derived from mammals, vertebrates, invertebrates, insects, bacteria, plant and fungi. In the present invention, the cell line is preferably derived from a mammalian source, such as human, rat, mouse, hamster, monkey and the like. “Derived from” in connection with a cell line means that one or more cells from a particular organism or microorganism was (or were) isolated using conventional techniques. Thus, the cell line may be comprised of cells directly from the organism or the progeny of such original cells.

The cell line may be an immortalized (i.e., continuous) cell line, i.e., a cell line that has been transformed in a manner such that it is adapted to cell culture conditions and may be passaged many times without altering the basic cellular pathways of the cell. As used herein, “passaged,” “passaging” and the like refer to the process of maintaining a cell line in a tissue culture flask at sub-confluent levels. The technique for passaging cells is well known in the art and will vary from cell type to cell type.

The cell line may also be a primary cell line, i.e., one that has recently been obtained from an explant and that may be passaged a limited number of times before the cellular pathways begin to change or the cell line begins to die.

The cell line may be one that is or has been adapted to grow in suspension. Nonlimiting examples of suspension cell lines include hybridomas, myeloma cells and the like. The cell line may be one that is adapted to be grown on a substrate surface. Nonlimiting examples of cell lines that are grown on a substrate surface include fibroblasts, such as 3T3 cells; epithelial cells, such as primary keratinocytes; and certain organ-derived cell lines, such as HEK-293 cells.

An “expression profile” means the pattern of expression of polypeptides that is unique to a cell line. The “expression profile” may be “generated” using any art recognized technique suitable for identifying specific polypeptides that are expressed, or mRNA transcripts that are transcribed, in a particular cell line. Such techniques include genomic and proteomic methods, including for example, the screening of arrays (micro- or macro-), high throughput screening of proteins separated using two-dimensional gel electrophoresis with a panel of antibodies and the use of a library of primers to screen for transcript amplification.

Identifying molecules, in general, or biomolecules, in particular, for further testing, in e.g. an end-point assay, from an expression profile is accomplished using art recognized methods. Such methods include detecting hybridization events by directly labeling a polynucleotide probe with a moiety that is detectable. As used herein, a “moiety that is detectable” means a radioactive or non-radioactive label. Examples of radioactive labels include ³H or ³²P labels. Examples of non-radioactive labels include fluorescent dyes and soluble or insoluble signaling moieties that are capable of generating a detectable color, including enzymatic systems such as alkaline phosphatase (AP) and horseradish peroxidase (HPO).

The molecule may be any molecule that is capable of modulating the sequence or its expression or a cellular process affected by the detected sequence or its expression, and may be present in an amount sufficient to achieve the same. Examples of such molecules include both organic and inorganic molecules. The molecules may be either synthetic or natural. Examples of organic molecules include, for example, natural or synthetic growth factors, cytokines, hormones, adhesion molecules, enzymes, biomolecules, and other related molecules. Examples of inorganic molecules include, for example, salts and minerals, such as for example, those that complex with cell receptors to modulate a detected sequence or its expression or cellular process affected by the same. “Biomolecule” means any biologically active molecule or small molecule that conveys an advantage to a cell in culture.

“Conveying an advantage,” “convey an advantage” or other similar phrases means that the biomolecule, when added to a cell line in a culture medium, enhances a biologically significant cellular process of that cell line in a way that is measurable using an end-point assay compared to a control medium that does not contain the biomolecule. There are many end-point assays that are well known in the art fordetermining how a particular biomolecule effects a particular cellular pathway and all such assays are within the scope of the present invention. Representative examples of such end-point assays include:

| | |
|---|---|
| Proliferation | (See e.g. Freshney, R. I. Culture of Animal Cells: a |
| assays - | Manual of Basic Technique. Fourth edition. Wiley-Liss, |
| | New York, 2000, which discloses a number of assays |
| | for determination of proliferative effects on cells (manual |
| | counting, DNA content assays, protein content assays, |
| | etc.); see also the resazurin assay described in more |
| | detail below); |
| Adhesion | (See Freshney, R. I. et al., supra, which also describes |
| Assays - | assays for plating efficiency, which is a function of |
| | adhesion); |
| Production | (See S. Peppers et al. (2001) Performance-Optimized |
| Assays - | Hybridoma Medium: Replacing Serum and Other |
| | Animal-Derived Components, Life Science Quarterly, |
| | Sigma-Aldrich Technical Application Newsletter, volume |
| | 2(2), which describes a typical production assay for the |
| | production of IgG in hybridoma lines); and |
| Differentiation | (See Klug, C. A. and Jordan, C. T. Hematopoietic Stem |
| Assays - | Cell Protocols in Methods in Molecular Medicine. |
| | Humana Press, Totowa, NJ. 2002, which describes |
| | many assays for determining the differentiative state of |
| | hematopoietic stem cells (flow cytometry, colony |
| | assays, etc.)). |

Each of the documents identified above is hereby incorporated by reference as if recited in full herein. Other end-point assays within the scope of the present invention include post translational modification assays, infection assays, apoptosis assays, paracrine control assays and immortalization assays.

The biomolecule may also be a ligand. “Ligand” means one member of a ligand/anti-ligand binding pair. The ligand may be, for example, one of the nucleic acid strands in a complementary, hybridized nucleic acid duplex binding pair; an effector molecule in an effector/receptor binding pair; or an antigen in an antigen/antibody; or substrate-enzyme complex or antigen/antibody fragment binding pair.

“Anti-ligand” means the opposite member of a ligand/anti-ligand binding pair. The anti-ligand may be the other of the nucleic acid strands in a complementary, hybridized nucleic acid duplex binding pair; the receptor molecule in an effector/receptor binding pair; or an antibody or antibody fragment molecule in antigen/antibody or antigen/antibody fragment binding pair, respectively.

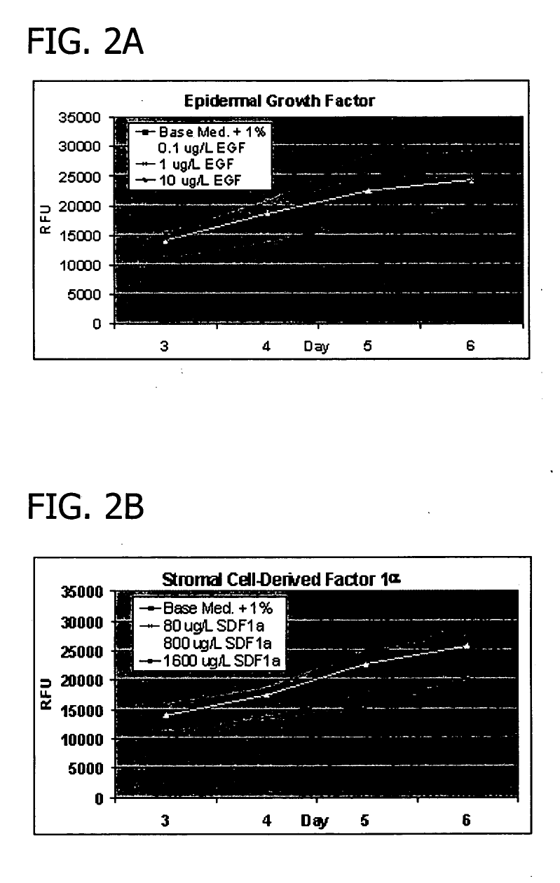

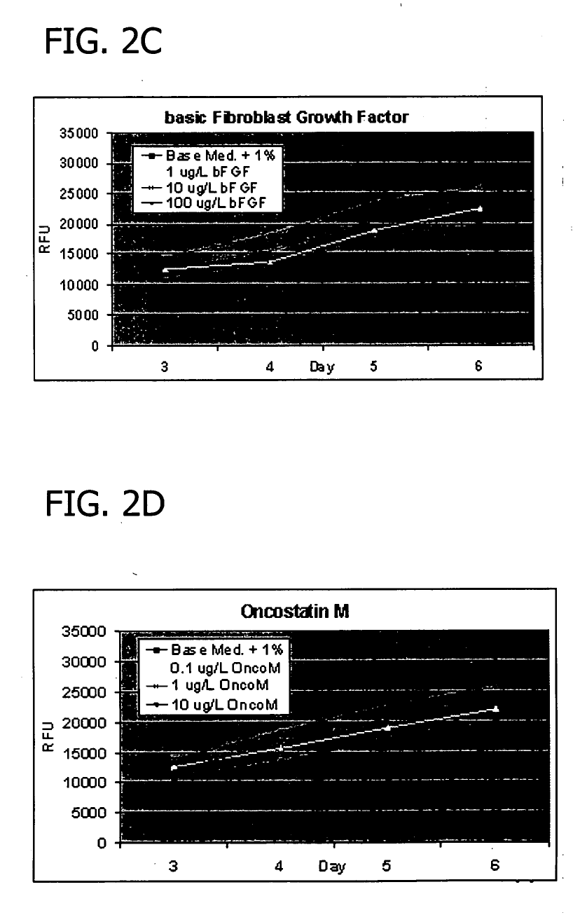

Non-limiting examples of biomolecules of the present invention are set forth in FIGS. 2, 5 and 7. A “set of biomolecules” means one or more biomolecules. In the present invention, the biomolecules may be selected from among several general classes of compounds, including: agonists, antagonists, ions, growth factors, cytokines, hormones, adhesion molecules and related molecules, extracellular matrix molecules, proteases, protease inhibitors, other cell surface receptors, enzymes, transcription factors, deoxyribozymes and ribozymes.

In the present invention, “small molecule” means a small organic or bio-organic molecule that, when added to a cell line in a culture medium, has a measurable effect in an end-point assay compared to a control medium that does not contain the small molecule.

Nonlimiting examples of growth factors include: platelet derived growth factor (PDGF), epidermal derived growth factor (EGF), fibroblast growth factor (FGF, including aFGF and bFGF) transforming growth factor (TGF, including TGF-α and TGF-β), NGF (nerve growth factor), insulin-like growth factor (IGF) and thrombopoietin (TPO).

Nonlimiting examples of cytokines include: interferon (IFN, including IFN-α and IFN-β), tumor necrosis factor (TNF), human growth hormone (HGH), Fas and interleukin (IL, including IL-1 through IL-15). Nonlimiting examples of hormones include insulin.

Nonlimiting examples of cell adhesion molecules include four general families: cadherins and catenins, immunoglobulin-like adhesion molecules, integrins and selectins. The integrin family includes: ITGA1 (integrin α1), ITGA2 (integrin α2/LFA1β), ITGA2B (integrin α2β), ITGA3 (integrin α3), ITGA4 (integrin α4/VLA-4), ITGA5 (integrin α5), ITGA6 (integrin α6), ITGA7 (integrin α7), ITGA8 (integrin α8), ITGA9 (integrin α9), ITGA10 (integrin α10), ITGA11 (integrin α11), ITGAL (integrin αL/LFA1α/CD11A), ITGAM (integrin αM), ITGAV (integrin αV), ITGAX (integrin αX), ITGB1 (integrin β1), ITGB2 (integrin β2), ITGB3 (integrin β3/CD61), ITGB4 (integrin β4), ITGB5 (integrin β5), ITGB6 (integrin β6), ITGB7 (integrin β7) and ITGB8 (integrin β8).

The Ig-like adhesion family includes: CEACAM5 (CEA), DCC, ICAM1, MICA (MUC-18), NCAM1, NRCAM, PECAM1 and VCAM1.

The cadherin and catenin family includes: CDH1 (E-cadherin), CTNNA1 (catenin α), CTNNAL1 (catenin α like-1), CTNNB1 (catenin β), CTNND1 (catenin δ1) and CTNND2 (catenin δ2).

The selectin family includes: SELE (ELAM-1/E-selectin), SELL (L-selectin) and SELP (P-selectin). Other related genes include CD44 and CNTN1.

The extracellular matrix protein family includes: CAV1 (caveolin-1), COL18A1 (LOC51695/endostatin), COLL A1, COL4A2, ECM1, FGB (fibrinogen β), FN1 (fibronectin-1), LAMB1 (laminin B1), LAMC1 (laminin B2), SPARC, SPP1 (OPN, osteopontin), THBS1 (TSP-1), THBS2 (TSP-2), THBS3 (TSP-3) and VTN (vitronectin).

The protease family includes matrix metalloproteinases, serine proteinases, cysteine proteinases and other related genes. Matrix metalloproteinases include: ADAMTS1 (Meth 1), ADAMTS8 (Meth 2), MMP1 (collagenase-1), MMP2 (gelatinase A), MMP3 (stromelysin-1), MMP7 (matrilysin), MMP8 (neutrophil collagenase), MMP9 (gelatinase B), MMP10 (stromelysin-2), MMP11 (stromelysin-3), MMP12 (macrophage elastase), MMP13 (collagenase-3), MMP14 (MT1-MMP), MMP15, MMP16, MMP17, MMP20 (enamelysin) and MMP24, MMP26. Serine proteinases include: CTSG (cathepsin G), PLAT (tPA), PLAU (uPA), PLAUR (uPAR) and TMPRSS4. Cysteine proteinases include: CASP8, CASP9, CST3 (cystatin C), CTSB (cathepsin B) and CTSL (cathepsin L). Other related genes to the proteinase family include: CTSD (cathepsin D), HPSE (heparanase) and MGEA5 (meningioma associated hyaluronidase).

The Protease inhibitor family includes: SERPINB2 (PAI-2), SERPINB5 (maspin), SERPINE1 (PAI-1), TIMP1, TIMP2 and TIMP3.

All of these molecules and families of molecules are biomolecules that have been implicated as participants in biologically significant cellular processes e.g. regulating cell division, cell growth, cell metabolism and/or adhesion, including cell-cell, cell-extracellular matrix and cell-substrate adhesion. Accordingly, identifying whether a particular cell line expresses one or more of these biomolecules is the first step in designing a cell culture medium according to the present invention. This first step may be accomplished using, e.g., an array containing a biopolymer that encodes a fragment of each such molecule.

Once a set of biomolecules that is expressed by a cell line is identified, the biomolecules and/or other biomolecules that are known to interact with same are tested, one at a time or in groups, to determine what effect they have, if any, on a biologically significant cellular process using one of the end-point assays set forth above. For example, in the present invention, a biomolecule that increases proliferation, relative to a control without such biomolecule, as measured in the resazurin assay set forth in the examples below is said to have a “measurable effect,” namely to “enhance cell growth or proliferation” and is considered to be a “positive biomolecule.”

Advantageously, the method of formulating a cell culture medium according to the present claims may be performed by detecting a sequence, such as, for example, an expressed amino acid sequence, from a single cell. The method does not require the comparison of a detected sequence from one cell to that of another, such as, for example, the comparison of an expressed amino acid sequence of one cell to an expressed amino acid sequence of another cell or cell line or the comparison of the effect of the molecule upon different cell lines or different culture conditions, whether by multiple microarray analyses or otherwise. While such a comparison of two different cells, whether the cells be different cell types or the same cell types subjected to different conditions, may be used to formulate a cell culture medium according to certain embodiments of the invention, such a comparison is not necessary or required.

The present invention encompasses both designing a cell culture medium from scratch or modifying an existing basal medium to support a cell line in a pre-defined manner. Accordingly, “formulating a cell culture medium” means designing a medium using the positive biomolecules identified in one or more of the end-point assays, e.g., the resazurin assay. More commonly, “formulating a cell culture medium” will mean modifying a basal medium by adding one or more positive ligands at a time and evaluating the modified basal media's ability to support a cell line of interest in a pre-defined manner. The process of adding one or more positive biomolecules and determining whether the modified medium is sufficient to support a cell line in the predefined manner is repeated, if necessary, until the modified medium is able to support the cell line in the pre-defined manner.

As used herein, “supporting a cell line in a pre-defined manner” or other similar phrases means that those biomolecules identified as positive in an end-point assay when added to a cell culture medium, e.g. a basal medium, will facilitate the cell line's survival in the medium and/or cause the cell line to behave or to exhibit characteristics desired by an investigator, e.g., growing and/or proliferating in a low or serum-free medium, having increased adhesion to other cells or substrate surfaces, increased production of a cellular byproduct, inducing differentiation, etc.

For example, in one embodiment, a modified medium is sufficient to support a cell line in a pre-defined manner if the modified basal medium or medium designed from scratch in accordance with the present methods is sufficient to maintain a particular cell line in a proliferative condition. For purposes of the present invention, a “proliferative condition” is one that is at least 50% of, preferably greater than 75% of, such as, at least about 90% of a control (cell line grown in recommended medium, including serum) using the resazurin assay set forth in the examples below.

“Basal medium” means a cell culture medium containing essential salts and amino acids in a buffered aqueous solution designed to support a cell line. Examples of commercially available basal medium include MCDB 153, Eagle's Basal Medium, Minimum Essential Medium, Dulbecco's Modified Eagle's Medium, Medium 199 (M199), Nutrient Mixtures Ham's F-10 and Ham's F-12, RPMI-1640. Such basal medium may also be supplemented with L-glutamine and/or various antibiotics, including for example penicillin and/or streptomycin according to guidelines for a particular cell line published by, for example, the American Type Culture Collection (ATCC) (Manassas, Va.).

A basal medium typically is serum-free but may be supplemented with serum. As used herein, “serum” is that component of the blood that is derived from clotted whole blood or plasma, which has been heat-inactivated (i.e., complement inactivated serum), although heat inactivation is not required. Serum may be obtained from various sources including calf and equine, most commonly fetal calf. Fetal calf serum is commercially available from a variety of sources including Sigma Aldrich Corp. (Cat No. F2442).

A “biopolymer” is a polymer composed of amino acids or nucleic acids. Thus, the phrases “nucleic acid sequence” or “polynucleotide” refer to a single or double-stranded polymer of deoxyribonucleotide or ribonucleotide bases read from the 5′ to the 3′ end. It includes chromosomal DNA, cDNA, mRNA, self-replicating plasmids, infectious polymers of DNA or RNA and DNA or RNA that performs a primarily structural role. It also includes nucleic acid analogs, such as for example peptide nucleic acid (PNA). It further includes both coding and non-coding nucleic acids, such as for example, introns, regulatory sequences, or housekeeping genes or nucleic acid sequences.

The terms “polypeptide,” “polypeptide sequence,” “amino acid,” and “amino acid sequence” are used interchangeably herein, and mean an oligopeptide, peptide, polypeptide, or protein sequence, or a fragment of any of these, as well as naturally occurring or synthetic molecules. In this context, “fragment” refers to fragments of any of the polypeptides defined herein which are at least about 30 amino acids in length. Where “amino acid sequence” is recited herein to refer to an amino acid sequence of a naturally occurring protein molecule, “amino acid sequence” and like terms are not meant to limit the amino acid sequence to the complete native amino acid sequence associated with the recited protein molecule.

Typically, when “biopolymer” is used herein in conjunction with an “array,” “microarray” or “macroarray” unless otherwise indicated, the biopolymer is a polypeptide fragment, an antibody or antibody fragment or a polynucleotide. The polynucleotide may be an mRNA or a cDNA sequence of at least about 100 nucleotides in length, preferably about 250 nucleotides in length. The polynucleotide may also be a shorter oligonucleotide. Polynucleotides of different lengths may be used and such lengths are readily determined by one skilled in the art with reference to well established procedures of array construction. The biopolymers either encode or are complementary to polynucleotide sequences that encode fragments of polypeptides of known function, such as those molecules involved in cell proliferation.

As used herein, an “antibody” means a protein that binds specifically to an epitope. The antibody may be polyclonal or monoclonal. The antibody may also be single chain (recombinant) antibodies, “humanized” chimeric antibodies, and immunologically active fragments of antibodies (e.g., Fab and Fab′ fragments). Such Fab fragments may be prepared in accordance with, e.g., the method of Huse et al., Science 246, 1275-1281.

When the array selected for use in the present methods is an antibody array, the polypeptides that are contacted with the array are derived from the cell line for which the cell culture medium is being designed. The polypeptides are derived from the cell line using conventional techniques, and may be used as e.g., whole cell extracts, homogenates, etc. Alternatively such polypeptides may be partially purified to remove non-protein contaminants.

The polypeptides may be labeled, using conventional labeling processes, such as metabolic labeling with, e.g., ³⁵S or ³H. The polypeptides may be detected using other direct or indirect labeling techniques, both radioactive and non-radioactive.

“Distinct biopolymers”, as applied to the biopolymers forming an array, means an array member which is distinct from other array members on the basis of a different biopolymer sequence, and/or different concentrations of the same or distinct biopolymers, and/or different mixtures of distinct or different-concentration biopolymers. Thus an array of “distinct polynucleotides” means an array containing, as its members, (i) distinct polynucleotides, which may have a defined amount in each member, (ii) different, graded concentrations of given-sequence polynucleotides, and/or (iii) different-composition mixtures of two or more distinct polynucleotides.

An “array,” means an organized arrangement of distinct biomolecules immobilized on substrates made of, e.g., nylon membrane, glass, plastic, silicon or any other high-modulus material. An “array” includes both macroarrays and microarrays. A “microarray” is an array of regions having a density of discrete regions of at least about 100/cm², and preferably at least about 1000/cm². The regions in a microarray have typical dimensions, e.g., diameters, in the range of between about 10-250 μm, and are separated from other regions in the array by about the same distance. The relative density and dimensions of such discrete regions in a “macroarray” are typically greater than that of a “microarray”.

An “array of regions on a solid support” is a linear or two-dimensional array of preferably discrete regions, each having a finite area, formed on the surface of a solid support.

In the present invention, the microarray may be any suitable microarray that contains distinct biopolymers encoding fragments of as many participants in biologically significant cellular processes as possible. Distinct biopolymers of the present invention include polynucleotides encoding a fragment of a polypeptide selected from the following families of molecules involved in cell proliferation: intracellular receptors, cell-surface receptors, enzymes, growth factors, cytokines, interleukins, transcription factors, hormones, adhesion molecules, cadherins and integrins. The biopolymers may also encode fragments of polypeptides selected from molecules involved in the following cellular processes: cell division, cell growth, cell metabolism and adhesion.

The arrays used in the present invention may be obtained commercially, such as for example, the Takara microarray set forth in the examples. It may, however, be necessary to utilize multiple arrays from commercially available sources in order to cover a broader range of molecules involved in cell proliferation.

Preferably, an array according to the present invention is used. In this array, which is preferably a microarray, at least the following biopolymers encoding fragments of the following participants in biologically significant cellular processes (set forth in Table 1) are arrayed on a suitable substrate surface:

TABLE 1

| Family | |
|---|---|
| Growth Factors | Molecule |
| | PDGF |
| | EGF |
| | aFGF |
| | bFGF |
| | TGF-α |
| | TGF-β |
| | NGF |
| | IGF |
| | TPO |
| Cytokines | IFN-α |
| | IFN-β |
| | TNF |
| | Fas |
| | IL-1 through IL-15 |
| Hormones | insulin |
| | human growth hormone |
| Cell Adhesion Molecules | |
| Integrins | ITGA1 |
| | ITGA2 |
| | ITGA2B |
| | ITGA3 |
| | ITGA4 |
| | ITGA5 |
| | ITGA6 |
| | ITGA7 |
| | ITGA8 |
| | ITGA9 |
| | ITGA10 |
| | ITGA11 |
| | ITGAL |
| | ITGAM |
| | ITGAV |
| | ITGAX |
| | ITGB1 |
| | ITGB2 |
| | ITGB3 |
| | ITGB4 |
| | ITGB5 |
| | ITGB6 |
| | ITGB7 |
| | ITGB8 |
| Ig-Like Adhesion Molecules | CEACAM5 (CEA) |
| | DCC |
| | ICAM1 |
| | MICA (MUC-18) |
| | NCAM1 |
| | NRCAM |
| | PECAM1 |
| | VCAM1 |
| Cadherins And Catenins | E-cadherin |
| | catenin α |
| | catenin α like-1 |
| | catenin β |
| | catenin δ1 |
| | catenin δ2 |
| Selectins And Related | ELAM-1/E-selectin |
| Genes | L-selectin |
| | P-selectin |
| | CD44 |
| | CNTN1 |
| Extracellular Matrix Proteins | CAV1 |
| | COL18A1 |
| | COL1A1 |
| | COL4A2 |
| | ECM1 |
| | fibrinogen β |
| | fibronectin-1 |
| | laminin B1 |
| | laminin B2 |
| | SPARC |
| | SPP1 |
| | THBS1 |
| | THBS2 |
| | THBS3 |
| | vitronectin |
| Proteases | |
| Matrix Metalloproteinases | Meth 1 |
| | Meth 2 |
| | collagenase-1 |
| | gelatinase A |
| | stromelysin-1 |
| | matrilysin |
| | neutrophil collagenase |
| | gelatinase B |
| | stromelysin-2 |
| | stromelysin-3 |
| | macrophage elastase |
| | collagenase-3 |
| | MT1-MM |
| | MMP15 |
| | MMP16 |
| | MMP17 |
| | enamelysin |
| | MMP24 |
| | MMP26 |
| Serine Proteinases | cathepsin G |
| | tPA |
| | uPA |
| | uPAR |
| | TMPRSS4 |
| Cysteine Proteinases | CASP8 |
| | CASP9 |
| | cystatin C |
| | cathepsin B |
| | cathepsin L |
| Related Protease Genes | cathepsin D |
| | heparanase |
| | meningioma associated |
| | hyaluronidase |
| Protease Inhibitors | PAI-2 |
| | maspin |
| | PAI-1 |
| | TIMP1 |
| | TIMP2 |
| | TIMP3 |

Any conventional method for making, e.g. a microarray containing at least the biopolymers identified in Table 1 may be used. Representative methods and substrates used in combinatorial array approaches are disclosed for example, by Southern et al. (U.S. Pat. Nos. 5,770,367, 5,700,637, and 5,436,327), Pirrung et al., (U.S. Pat. No. 5,143,854), Fodor et al. (U.S. Pat. Nos. 5,744,305 and 5,800,992), and Winkler et al. (U.S. Pat. No. 5,384,261).

Ink-jetting and other “drop-on-demand” devices are also available for the fabrication of biological and chemical arrays as shown by Brennan (U.S. Pat. No. 5,474,796), Tisone (U.S. Pat. No. 5,741,554), and Hayes et al. (U.S. Pat. No. 5,658,802).

A third category of arraying devices work by direct surface contact printing as described by Augenlicht (U.S. Pat. No. 4,981,783), Drmanac et al. (U.S. Pat. No. 5,525,464), Roach et al. (U.S. Pat. No. 5,770,151), and Brown et al. (U.S. Pat. No. 5,807,522).

Another category of arraying device is made with advanced machining technologies such as an electronic discharge machine (EDM), thereby providing for precise sample uptake and delivery. (See Martinsky, U.S. Pat. No. 6,101,946). Each of the patents summarized above is hereby incorporated by reference as if recited in full herein.

A “pool” or “plurality” of polynucleotide probes are used to identify which biopolymers are expressed by a cell line. The polynucleotide probes are derived from or generated from a cell line for which a medium is to be designed or optimized using conventional methods, such as the method set forth in the examples. The polynucleotide probes, which are preferably mRNA or cDNA, are engineered to be detectable by any conventional means. Thus, as set forth above, the probes may be made to include a radioactive label, such as ³H or ³²P. Alternatively, the probes may be end-labeled with a unique capture sequence that is recognized by an oligonucleotide probe that contains a detectable moiety, such as a fluorescent or other colored dye or an enzyme that produces a detectable signal such as the alkaline phosphatase or horseradish peroxidase detection systems. Still further, the nucleotide may be labeled, on the phosphate, base or sugar moiety, with a directly detectable label or with a ligand that will bind to an anti-ligand labeled with a detectable signal.

The polynucleotide probes are “contacted” with the biopolymers on the microarray. This means that the polynucleotide probes, suspended in an appropriate buffer, such as the buffer described in the examples, are dispersed over the microarray under conditions sufficient to allow specific hybridization of the probes to any biopolymer on the microarray with a complementary sequence.

The phrase “specific hybridization” refers to the binding, duplexing, or hybridizing of a molecule only to a particular nucleotide sequence under stringent hybridization conditions when that sequence is present in on the microarray.

The phrase “stringent hybridization conditions” refers to conditions under which a probe will hybridize to its target sequence, but to no other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Probes, “Overview of Principles of Hybridization and the Strategy of Nucleic Acid Assays” (1993). Generally, highly stringent conditions are selected to be about 5-10° C. lower than the thermal melting point (Tₘ) for the specific sequence at a defined ionic strength and pH. Low stringency conditions are generally selected to be about 15-30° C. below the Tₘ. The Tₘ is the temperature (under defined ionic strength, pH, and nucleic acid concentration) at which 50% of the probes complementary to the target hybridize to the target sequence at equilibrium (as the target sequences are present in excess, at Tₘ, 50% of the probes are occupied at equilibrium). Stringent conditions will be those in which the salt concentration is less than about 1.0M sodium ion, typically about 0.01 to 1.0M sodium ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. For selective or specific hybridization, a positive signal is at least two times background, preferably 10 times background hybridization.

The detection procedure used to identify the probes that hybridize to the array is not critical and may be selected by the researcher based on conventionally available detection methods as described above or in the examples. Briefly, the detection procedures may be detecting radiolabeled probes in a radioactive detection device, detecting fluorescent signals in a fluorescent reader adapted for reading arrays or detecting colored precipitates in an automated reader adapted for reading arrays. The hybridization and subsequent detection of the binding of a probe to a biopolymer on the array confirms that the cell line from which the probe is made expresses the molecule whose partial nucleotide sequence is immobilized on the array. Based on this result, e.g., a ligand (or other biomolecule) corresponding to the molecule partially encoded by the biopolymer is identified for further testing in e.g., the cell proliferation assay (or other end-point assay).

Generating the expression profile is not limited to using an array. Accordingly, the expression profile may be generated by making oligonucleotide primer sets designed to amplify the mRNA of molecules that participate in biologically significant cellular processes, e.g., cell proliferation, such as those molecules set forth in FIGS. 2, 5 and 7 and/or those molecules identified in Table 1. Additional suitable primer sets may be identified from other conventional libraries containing participants in significant cellular processes, such as for example, cell proliferation. Each primer set will be comprised of an oligonucleotide complementary respectively to the 3′ and 5′ termini of distinct biopolymers. Each oligonucleotide in the primer set will be of a length sufficient to allow transcription of a message. Typical oligonucleotide lengths will be from about 1040 nucleotides, preferably 20-24 nucleotides in length.

The primer sets are then used to amplify polynucleotide sequences, e.g. RNA obtained from a cell line for which medium design/optimization is desired. The polynucleotide sequences to be amplified may be reverse transcribed from RNA derived from the cell line for which the cell culture medium is being designed using e.g., the methods set forth in the Examples. The amplification takes place under standard PCR conditions using standard reagents, such as those identified in Sambrook et al., Molecular Cloning A Laboratory Manual, 2^{nd} Ed. (1989) pp. 14.14-14.21, which is hereby incorporated by reference as if recited in full herein.

The amplified transcripts for each primer set are then separated using agarose gel electrophoresis and identified. Those primer sets that amplify mRNA from the cell line identify molecules expressed by the cell line for which the corresponding biomolecule (or its ligand) may be tested for its ability to effect an end-point assay, e.g. to enhance proliferation of the cell line in a proliferation assay.

Within the scope of the present invention is an additional method for generating the expression profile utilizing proteomics methodologies. In this method, a protein sample is prepared from, e.g., a whole cell homogenate of a cell line for which medium design/optimization is desired. The protein homogenate is then separated using, e.g., conventional two-dimensional gel electrophoresis, whereby in one dimension, the proteins are separated by pH (isoelectric focusing), and in the second dimension, the proteins are separated by size and charge (electrophoresis). The respective protein spots may be transferred to, e.g., a solid substrate, such as nitrocellulose paper, for further processing (such as for example Western blotting).

A panel of antibodies, such as monoclonal antibodies, directed against molecules that participate in biologically significant cellular processes as defined above, e.g. in cell proliferation, are contacted with the separated proteins on, e.g., the Western blot. The protein spots to which the antibodies specifically bind are identified (using radioactive or non-radioactive means as described above) and the corresponding biomolecule (or ligand) to each protein is thus identified for further testing using the end-point assays for inclusion in the cell culture medium to be designed/optimized.

In the present invention, the medium that is designed and/or optimized is a low serum medium. As used herein, “low serum medium” means that the medium contains less than 10%(v/v or wt) of serum, preferably less than 7.5%(v/v or wt) of serum, more preferably less than 5%(v/v or wt) of serum, such as for example between 1%-3%(v/v or wt) of serum or less than 1%(v/v or wt) serum. Alternatively, the medium may be serum-free. By “serum-free,” it is meant that no amount of serum may be detected in the medium.

A further embodiment of the invention is a method for identifying biomolecules for use in designing a cell culture medium adapted to support a cell line in a pre-defined manner. In this method, a pool of polynucleotide probes are designed from the cell line for which the cell culture medium is to be designed. The probes are complementary to polynucleotide sequences that encode fragments of polypeptides expressed by the cell line as described in more detail above.

The probes are then contacted with at least one array containing biopolymers immobilized on a surface thereof as previously described. The hybridization of the probes to the biopolymers is detected and molecules expressed by the cell line are identified (i.e., they form an expression profile). Based on this expression profile, biomolecules corresponding to the expressed proteins are identified and selected as candidate components for use in designing a cell culture medium for the cell line.

A further embodiment of the invention is a method for preparing a serum-free cell culture medium that is sufficient to support a cell line. In this method, a pool of polynucleotide probes is generated. The probes are complementary to polynucleotide sequences that encode fragments of polypeptides expressed by the cell line. As described in more detail above, the probes are then contacted, under hybridizing conditions, with at least one array containing a plurality of biopolymers immobilized on the surface of the array, each biopolymer encoding a fragment of a distinct polypeptide that participates in a biologically significant cellular process, such as regulating cell growth and/or proliferation. Next, an expression profile is generated by detecting each polynucleotide probe that hybridizes to each biopolymer as set forth previously. Biomolecules are then selected, based on the expression profile, to be candidate components for a serum-free medium based on the function of the polypeptide partially encoded by the biopolymers to which a probe hybridized. As set forth above, each candidate component is tested to determine its effect on growth and/or proliferation of the cell line or other biologically significant cellular process using an end-point assay. Those candidate components that, e.g. increase or enhance growth and/or proliferation of the cell line are designated as “positive biomolecules.” The positive biomolecules are then added to a serum-free basal medium and evaluated using e.g., the resazurin assay for their ability to enhance the growth and/or proliferation of the cell line in the modified medium. If necessary, the previous step is then repeated until the modified medium will support the cell line.

Another embodiment of the invention is an array comprising at least one nucleic acid of, but less than the entire genome of, a Chinese hamster cell, the nucleic acid being immobilized on the surface of the array. The array may comprise a plurality of nucleic acids. The nucleic acid may be a polynucleotide encoding or regulating the expression of receptors, enzymes, cytokines, interleukins, transcription factors, hormones, adhesion molecules, cadherins, or integrins. Likewise, the nucleic acid may be a deoxyribozyme, a ribozyme, a microRNA, or a nucleic acid analog, such as for example a peptide nucleic acid (PNA). In a preferred embodiment, the array comprises a nucleic acid encoding or regulating the expression of receptors, enzymes, cytokines, interleukins, transcription factors, hormones, adhesion molecules, cadherins, or integrins, and a nucleic acid for Chinese hamster housekeeping genes. Such housekeeping genes include, for example, cytoplasmic actin, GAPDH (glyceraldehyde phosphate dehydrogenase), tubulin.

By way of example, the array may contain a nucleic acid of a Chinese hamster cell encoding a single or multiple cell-surface receptors, but will not contain the entire Chinese hamster genome. Alternatively, the array may contain a nucleic acid regulatory sequence or a non-coding region of a Chinese hamster ovary, but will not contain the entire Chinese hamster genome. In either example, the array could also comprise housekeeping genes from a Chinese hamster cell.

Alternatively, the array may also be a polypeptide array, comprising a polypeptide encoded by a nucleic acid from the Chinese hamster genome, or an antibody directed against such a polypeptide or the nucleic acid that encodes the polypeptide. The polypeptide may be the entire product encoded by a particular nucleic acid or merely a fragment thereof.

Another embodiment of the invention is directed to a method for preparing a cell culture medium, said method comprising contacting a polynucleotide with an array comprising a plurality of biopolymers immobilized on the surface of the array, the polynucleotide being derived from a cell or the complement thereof; detecting a bound pair formed between the polynucleotide and an immobilized biopolymer; selecting a molecule for inclusion in a cell culture medium based on the members of the detected bound pair; testing selected molecule to determine its effect on a cellular process of the cell; and formulating a cell culture medium to include the selected molecule.

Another embodiment of the invention is directed to method for preparing a cell culture medium, said method comprising: contacting a polypeptide with an array comprising a plurality of biopolymers immobilized on the surface of the array; detecting a bound pair formed between the polypeptide and an immobilized biopolymer; selecting a molecule for inclusion in a cell culture medium based on the members of the detected bound pair; testing selected molecule to determine its effect on a cellular process of the cell; and; and formulating a cell culture medium to include the selected molecule.

Cell culture medium made by any of the processes set forth herein are also encompassed within the scope of the present invention.

The following examples are provided to further illustrate certain of the methods and materials of the present invention. These examples are illustrative only and are not intended to limit the scope of the invention in any way.

### EXAMPLES

### Example 1

### Microarray Analysis

A. HEK-293 Cells

Materials and Methods

In the following examples, HEK-293 cells were used. The HEK-293 cell line is a permanent line of primary human embryonal kidney transformed by sheared human adenovirus type 5 (Ad 5) DNA. This cell line is commercially available from, e.g., ATCC(CRL-1573).

The microarray used herein was the Takara IntelliGene Cytokine CHIP (version 2.0) (Takara Bio Inc., Shiga, Japan). This microarray contains approximately 550 cDNA fragments (approximately 300 bp regions of each gene) arrayed and immobilized on a glass slide, which represent various human growth factors/cytokines and their receptors.

Isolation of mRNA

RNA was prepared from confluent cultures of HEK-293 grown in reduced serum conditions (MCDB 153+1% fetal bovine serum (FBS)). The HEK-293 cells were stored in 10 ml of RNAlaterä buffer at −20° C. until RNA isolation. RNAlater is an aqueous nontoxic solution that permeates cells to stabilize RNA (technical bulletin R0901, Sigma-Aldrich Corp., St. Louis, Mich.). RNA was isolated using a GenElute Direct mRNA Miniprep Kit (DMN-10/DMN-70, Sigma-Aldrich Corp., St. Louis Mich.) according to the instructions provided in the technical bulletin with minor modifications as required to accommodate samples in RNAlater.

Briefly, HEK-293 cells were pelleted by centrifugation at 820×g for 10 minutes. The cells were vortexed in lysis buffer containing proteinase K and filtered through a filtration column. The homogenized lysate was then incubated at 65° C. for 10 minutes for proteinase K digestion. The solution was then prepared for mRNA binding to oligo dT beads (Sigma Aldrich Corp., Cat No. 03131) by the addition of sodium chloride.

Oligo dT beads were added to the lysate solution and mixed. The bead/lysate solution was incubated for 10 minutes at room temperature to permit binding of polyA mRNA to the oligo dT beads. The bead/lysate solution was then diluted up to 2-fold with wash solution or lysis buffer/salt solution to permit subsequent pelleting of oligo dT beads. The oligo dT beads were then pelleted and washed several times with wash solution and low salt wash solution in a spin basket.

The RNA was then eluted in elution buffer at 65° C. The mRNA was subsequently concentrated by adding glycogen, 0.1 volume 3M sodium acetate, pH 5.2, 2.5 volumes 100% ethanol and precipitated overnight. The precipitated material was next pelleted by centrifugation at 4° C., and washed with 75% ethanol. Once again, the material was pelleted by centrifugation at 4° C. Finally, the pellet was dried and reconstituted in water.

Adding Genisphere 3DNA Capture Sequences To The cDNA

The mRNA isolated from the HEK-293 cells was reverse transcribed into complementary DNA, cDNA, as described in the product manual provided with the Genisphere® 3DNA™ Submicro EX Expression Array Detection Kit (catalog number A100782) (Haffield, Pa.), with the minor modification that SigmaSpin size exclusion columns (S5059, Sigma-Aldrich Corp.) were used in place of the Genisphere SCL spin columns. Two addition reactions were prepared for hybridization on a single microarray—one for Cy3 detection and another for Cy5 detection. Thus, normalized Cy3 and Cy5 signals were to ideally have a ratio of “1” for each microarray spot.

For each reaction, 1 μg of mRNA was incubated with the RT primer (Cy3 or Cy5) purchased from Genisphere for 10 minutes at 80° C. and then chilled on ice. The RT primer is an oligo dT primer, which contains a 5′ capture sequence complementary to either a Cy3 or Cy5 fluorescently tagged 3DNA reagent. The 3DNA reagent is a dendrimer containing approximately 375 fluorescent dyes (in this case either Cy3 or Cy5) per molecule. Superase-In™, an RNase inhibitor, was added, followed by the reverse transcription reaction mix (reverse transcriptase buffer, dNTPs, and reverse transcriptase enzyme).

The reaction was then heated for 2 hours at 42° C. followed by the addition of stop solution (0.5M NaOH, 50 mM EDTA), incubated at 70° C. for 10 minutes and then neutralized with 1 M Tris, pH 7.8. The Cy3 and Cy5 reactions were then pooled and unincorporated primers, dNTPs, salts, etc. were removed by purification over two SigmaSpin columns. The 3DNA capture sequence labeled-cDNA was then concentrated using a Microcon 30 spin column.

Pre-Hybridization and cDNA Hybridization

The microarray analysis of this example was performed in duplicate. The Takara Chips were pre-hybridized at 42° C. in approximately 30 ml of 5×SSC, 25% formamide, 0.1% sodium lauroylsarcosine, 1% bovine serum albumin for 1 hour in a screw top 4-slide holder (PAP jar, Evergreen Scientific) with rotation in a hybridization oven (Stovall Life Sciences, Inc.). The pre-hybridization step was followed by a water rinse and drying.

The microarray hybridization was conducted under a LifterSlipä coverslip (catalog number 22×251, Erie Scientific) that was previously washed/blocked in 0.5% SDS and rinsed with water. A LifterSlip is a coverslip that has printed bars along two opposite edges, which raises the coverslip over the sample to permit better solution kinetics. The hybridization solution was prepared by mixing the cDNA-dendrimer complex with 2×formamide-based hybridization solution (50% formamide, 8×SSC, 1% SDS, 4× Denhardt's solution), LNA™ dT blocker and Block-It™ human DNA (ID Labs, equivalent to Cot-1 DNA, i.e. repetitive sequence DNA). The LNA™ dT blocker contains locked nucleic acid nucleotides at key positions within the poly dT synthetic strand and is designed to block all poly A containing elements, including spotted poly dA sequences.

The hybridization solution was heated at 70° C. for 10 minutes, followed by 45° C. for 15 minutes and then applied to two pre-hybridized, pre-warmed (42° C.) Takara microarrays under an SDS washed LifterSlip. The arrays were incubated overnight in a humid chamber floated in a 42° C. water bath. Post-hybridization washes were performed by placing the microarrays in pre-warmed (55° C.) wash solution (2×SSC, 0.2% SDS), and incubating the array in a 55° C. hybridization oven with rotation for 10 minutes. This incubation step was followed by two 10 minute room temperature washes, wherein the first wash employed 2×SSC and the second 0.2×SSC. The arrays were then placed in 95% ethanol for 2 minutes and dried.

3DNA Detection

Detection of the cDNA-dendrimer complex was also performed using the Genisphere® 3DNA™ Submicro EX Expression Array Detection Kit (catalog number A100782). The 3DNA detection solution contained Cy3 and Cy53DNA capture reagents (warmed at room temperature, vortexed, centrifuged, warmed at 50° C. for 10 minutes and vortexed again to break up potential aggregates), 2×formamide-based hybridization solution (50% formamide, 8×SSC, 1% SDS, 4× Denhardt's solution), high-end differential enhancer (to help increase the differential between Cy3 and Cy5 samples run on the same array) and anti-fade reagent. The 3DNA detection solution was first heated at 75° C. for 10 minutes, then 50° C. for 15 minutes and applied to the arrays at 55° C. under an SDS washed LifterSlip. The arrays were then incubated for 2 hours in a humid chamber floated in a 50° C. water bath.

Next, a washing step was conducted by placing the arrays in pre-warmed (60° C.) wash solution (2×SSC, 0.2% SDS) and incubating the arrays in a 60° C. hybridization oven with rotation for 10 minutes. The arrays were subsequently washed again with 2×SSC and then with 0.2×SSC for 10 minutes each at room temperature. The arrays were then dried and immediately scanned on a ScanArray Express (PerkinElmer Life Sciences), a microarray laser scanner. Images from the Cy3 and Cy5 channels were selected for quantitation where the signals were maximal yet below saturation. Signals were normalized using the ScanArray Express software by normalizing to total signal. The ratios of Cy5/Cy3 signals for most spots were very close to “1.”

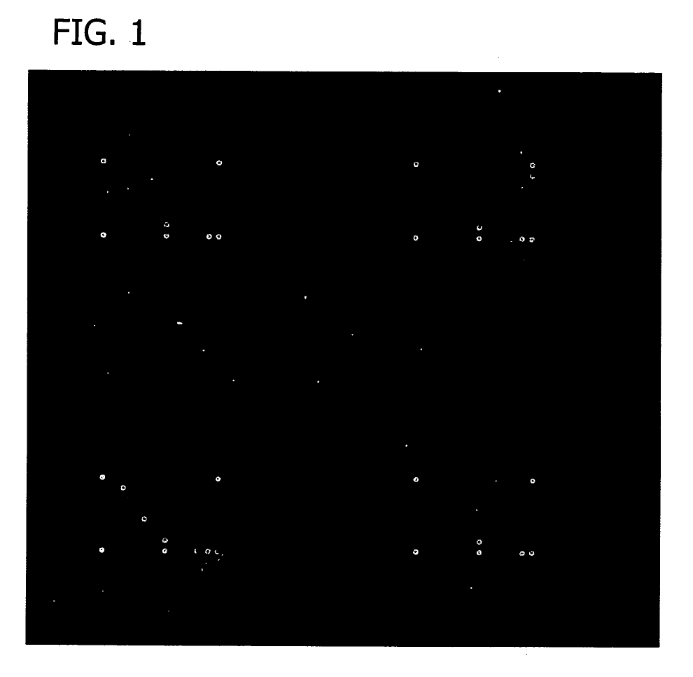

The image of a representative microarray generated in accordance with this example is shown in FIG. 1. Quantitation of the spots revealed positive expression for many genes. In order to facilitate the interpretation of the data obtained from the arrays, a limit based on background readings was established. Since both fluorophores (Cy3 and Cy5) labeled the identical RNA population, it was determined that if the sum of the relative fluorescence units (RFU) for both channels was greater than 200 (after background subtraction), then positive expression could be identified on that basis.

The positive genes that were found to be expressed in the HEK-293 cells, i.e. positive expression, were divided into various groups, including growth factor/cytokine receptors and cell adhesion molecules. Table 2 shows a list of growth factor/cytokine receptors (a corresponding ligands) expressed by the HEK-293 cells, which were identified by the microarray analysis and illustrated in FIG. 1. The corresponding ligand for each identified receptor is also provided. Thus, from one experiment, it was determined that the HEK-293 cells express 27 growth factor/cytokine receptors, which provided a starting point for formulating a serum-free medium. Of the 27 “positive” growth factor/cytokine receptors, 16 were selected for further testing.

TABLE 2

| Receptor | Ligand | Receptor | Ligand |
|---|---|---|---|
| AXL receptor tyrosine kinase | gas6 | neuropilin 1 | VEGF* |
| EGF receptor | EGF* | macrophage stimulating 1 receptor | MSP |
| chemokine (C-X3-C) receptor 1 | Fractalkine | PDGF receptor, α polypeptide | PDGF AB* |
| PDGF receptor, β polypeptide | PDGF AB* | nerve growth factor receptor | NGF* |
| interleukin 15 receptor, α | IL-15 | interleukin 11 receptor, α | IL-11* |
| interleukin 2 receptor, α | IL-2* | interleukin 10 receptor, β | IL-10* |
| interleukin 2 receptor, β | IL-2* | FGF receptor 4 | aFGF* |
| chemokine (C-C motif) receptor 2 | MCP1 | bone morphogenetic protein receptor, type II | BMP-2* |
| interleukin 2 receptor, γ | IL-2* | TGF, β receptor II | TGFβ* |
| interleukin 18 receptor 1 | IL-18 | FGF receptor 1 | bFGF* |
| colony stimulating factor 1 receptor | CSF-1 | chemokine (C-X-C motif), receptor 4 | SDF1α* |
| oncostatin M receptor | OSM* | interferon γ receptor 1 | IFNγ* |
| interleukin 4 receptor | IL-4* | interferon γ receptor 2 | IFNγ* |
| vitamin D3 receptor | Vitamin D3* | | |

*denotes growth factors tested in vitro

High Throughput Cell Culture Assay

To evaluate whether the “positive” growth factors identified in the microarray analysis actually had an effect on HEK-293 growth/proliferation, a high throughput cell culture assay was developed to measure proliferation utilizing resazurin (Sigma-Aldrich Corp.) (Cat No. TOX-8 or 7017). Resazurin is a metabolic dye which is converted to a fluorescent product. The greater the amount of the fluorescent product that is generated, the more metabolism, which directly correlates with cell number.

In this assay, resazurin was added to HEK-293 cells plated in each well of a 24-well assay plate containing a test medium containing one of the 16 ligands corresponding to the positive growth factors/cytokines and incubated at 37° C. After a 30 minute incubation, the plate was analyzed on a standard fluorescence plate reader. A higher RFU value from the plate reader translates to a higher cell density in the wells.

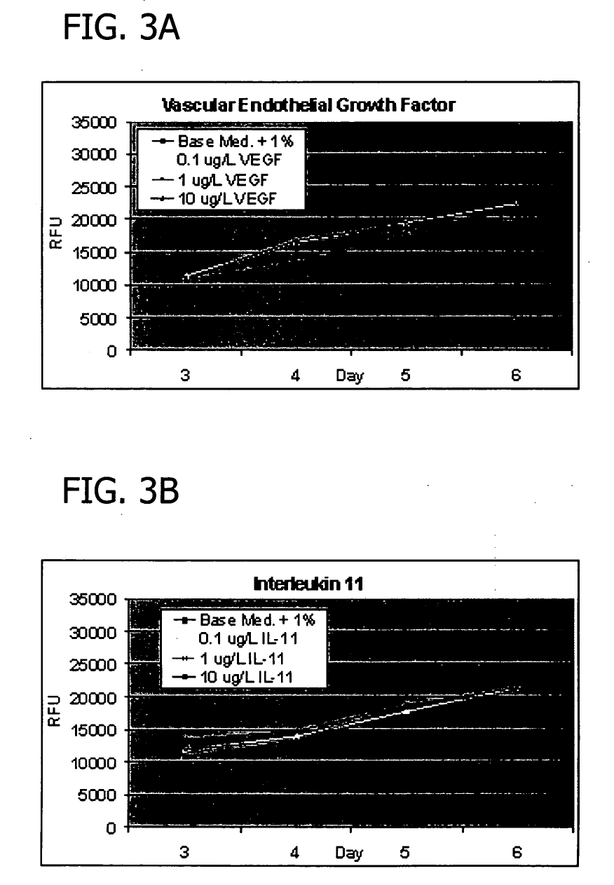

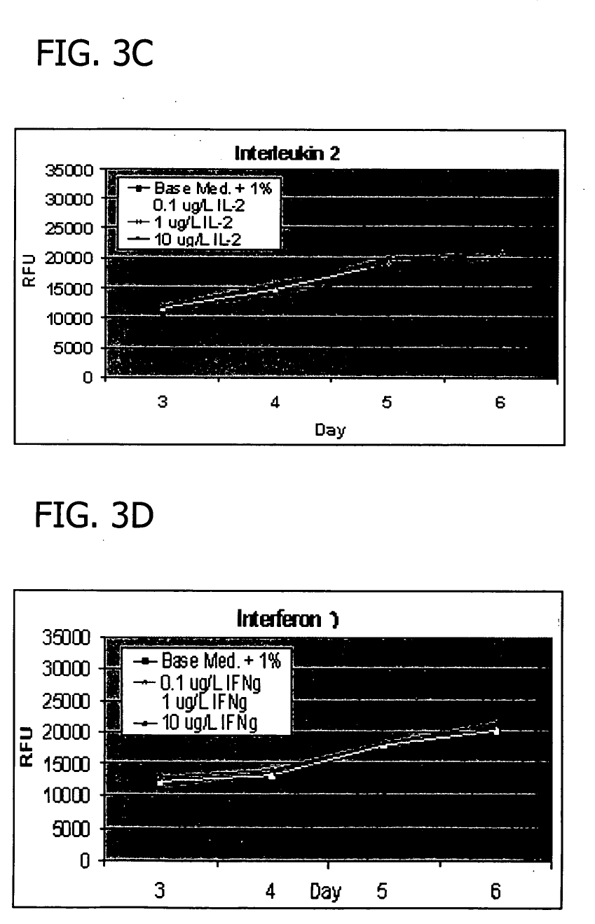

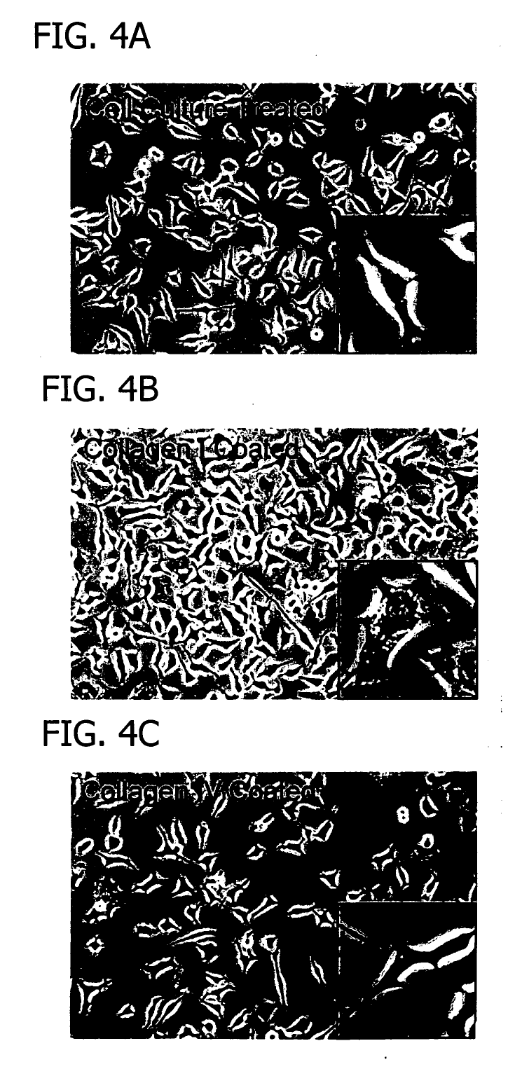

Briefly, the 16 ligands corresponding to positive growth factor/cytokine receptors were tested in the resazurin assay, each ligand at three different concentrations. The graphical representations of the RFU values observed for each factor, shown in FIGS. 3 and 4, include the base medium (with 1% FBS) shown as the pink line. The other 3 lines on each graph represent the three (3) concentrations of growth factor tested in the assay. Some of the factors exhibited a positive effect (FIG. 2A-2D), which indicates that they were considerably better than the base medium. In particular, four components exhibited a significant effect: epidermal growth factor (EGF), basic fibroblast growth factor (bFGF), oncostatin M and stromal cell-derived factor 1a (SDF1α). Some of the factors had an intermediate effect (not shown), which means that they grew only slightly better than the base medium. Finally, some factors had no effect on the proliferation of the HEK-293 culture (FIG. 3A-3D).

The boost in proliferation, which accompanies the “positive” growth factors and cytokines, should allow an investigator to reduce or eliminate the amount of serum added to a basal medium. Since SDF1α and oncostatin M are factors that certainly would not have been tested with the HEK-293 cell line based only on what is known in the public literature, the utility of the foregoing methods and materials is significant. That is, in this example, it was clear that the use of the expression profiling was indispensable; and it is highly unlikely that the key components for this medium would have otherwise been identified.

Using another group of proteins expressed in the HEK-293 cells based on the microarray analysis, we identified 20 cell adhesion related molecules, which are listed in Table 3. Table 3 is a table containing a list of “positive” adhesion related proteins identified from the microarray of 1. HEK-293 cells are an adherent cell line. Thus, the adhesion properties of these cells are important to consider when designing cell culture medium, especially low or serum-free medium because, in general, the ability of cells to attach to substrates decreases as the levels of serum in a medium decrease.

TABLE 3

| Protein | Function |
|---|---|
| integrin, α8 | Cell to Substrate interaction |
| integrin, α9 | Cell to Substrate interaction |
| integrin, α3 | Cell to Substrate interaction |
| integrin, α1 | Cell to Substrate interaction |
| integrin, β5 | Cell to Substrate (vitronectin) interaction |
| integrin, α7 | Cell to Substrate interaction |
| integrin, αV | Cell to Substrate (vitronectin) interaction |
| integrin, αE | Cell to Substrate (E-cadherin) interaction |
| E-cadherin | Cell to Cell interaction; epithelial |
| P-cadherin | Cell to Cell interaction; placental |
| OB-cadherin | Cell to Cell interaction; osteoblast |
| N-cadherin | Cell to Cell interaction; neuronal |
| vascular cell adhesion | Binds integrins α4β1 and α4β7 |
| molecule 1 | |
| intercellular adhesion | Binds integrin αLβ2 |
| molecule 2 | |
| activated leucocyte cell | Binds CD6 on leucocytes |
| adhesion molecule | |
| matrix metalloproteinase 9 | Cleaves collagen IV |
| matrix metalloproteinase 10 | Cleaves collagen IV |
| matrix metalloproteinase 15 | Cleaves fibronectin |
| tissue inhibitor of | Inhibits MMP2 (cleavage of collagen I,IV) |
| metalloproteinase 2 | |
| tissue inhibitor of | Inhibits MMP3 (cleavage of collagen III,IV) |
| metalloproteinase 3 | |

Based on the profile of adhesion molecules shown in Table 3, the ability of the HEK-293 cells to attach to a variety of permissive substrates was examined. Several observations were made that correlated with the information gathered through the microarray analysis. For example, it was found that HEK-293 cells did not adhere well to plates coated with collagen IV. This was explained by the expression of matrix metalloproteinase 9 (MMP9) protein, which serves to cleave collagen IV, in the HEK-293 cell line. Thus, the MMP9 protein most likely degraded the collagen IV coating on the plate, thus leaving an unacceptable substrate for the cells.

FIG. 4 shows HEK-293 cells grown on a normal non-coated 6-well plate (A), a plate coated with collagen I (B) and a plate coated with collagen IV (C). Cells attached and proliferated on the plate coated with collagen 1, possibly due to the presence of integrin a1, which with integrin b1, can bind collagen 1. Cells cultured on the collagen IV coated plates exhibited attachment in the same manner as the uncoated plates. This would be consistent with the expression of MMP9, having degraded the collagen IV.

Table 4 is a table containing a list of other proteins that may play a role in HEK-293 growth and/or proliferation.

TABLE 4

| Protein | Function |
|---|---|
| insulin-like growth | Potent growth factor |
| factor 1 (IGF1) | |
| insulin-like growth | Potent growth factor |
| factor 2 (IGF2) | |
| IGF binding protein 5 | Involved in the regulation of IGF function |
| IGF binding protein 6 | Involved in the regulation of IGF function |
| IGF binding protein 7 | Involved in the regulation of IGF function |
| EphA1 | Tyrosine kinase receptor involved |
| | in pattern formation |
| EphA2 | Tyrosine kinase receptor involved |
| | in pattern formation |
| EphB2 | Tyrosine kinase receptor involved |
| | in pattern formation |
| EphB4 | Tyrosine kinase receptor involved |
| | in pattern formation |
| EphB6 | Tyrosine kinase receptor involved |
| | in pattern formation |
| ephrin-B1 | Ligand for Eph receptors also involved |
| | in pattern formation |
| cyclin A2 | Involved in cell cycle regulation |
| cyclin E1 | Involved in cell cycle regulation |
| cell division cycle 2 | Involved in cell cycle regulation |
| cyclin-dependent kinase 2 | Involved in cell cycle regulation |
| endoglin | Glycoprotein involved in adhesion |
| | and proliferation |
| neuregulin 1 | Involved in cell signaling via |
| | tyrosine kinase receptors |

Serum-Free HEK-293

Using the information gathered from the microarray analysis and the in vitro tests, above, a serum free medium is made with the following components:

| MCDB 153 | |
|---|---|
| L-glutamine | 2 mM |
| Earle's BSS | adjusted to contain |
| | 1.5 g/L sodium |
| | bicarbonate |
| non-essential amino acids | 0.1 mM |
| sodium pyruvate | 1.0 mM |
| EGF | 10 μg/L |
| SDF1a | 1600 μg/L |
| bFGF | 100 μg/L |
| OncoM | 10 μg/L |
| Collagen I | coating for substrate |

It is expected that such a medium will support the growth and proliferation of HEK-293 in the absence of any serum. This is expected to be confirmed using the resazurin assay, wherein if the cell line grown in the serum-free medium proliferates at a rate that is at least 75% that of a control with the same cells grown in MCDB 153 medium supplemented with 10% FBS under otherwise identical conditions for the same amount of time, the serum-free medium is deemed able to support the cell line.

B. WI-38 Cells

In order to test the possibility of using the method as applied to HEK-293 cells to identify pathways in other cell lines, the normal human fibroblast, WI-38 was chosen. These cells are typically grown in a base medium containing anywhere from 3-10% FBS.

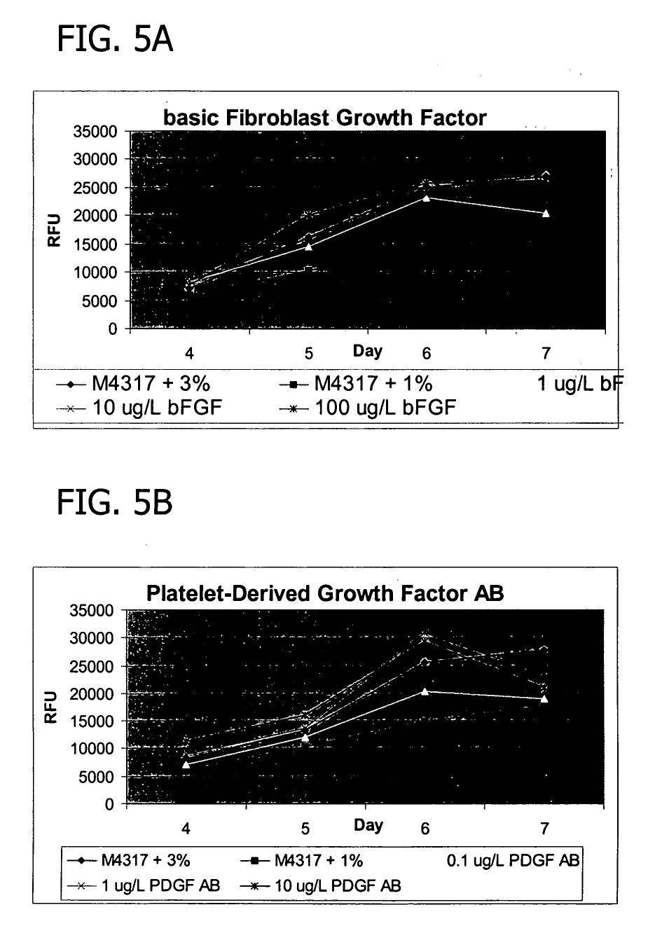

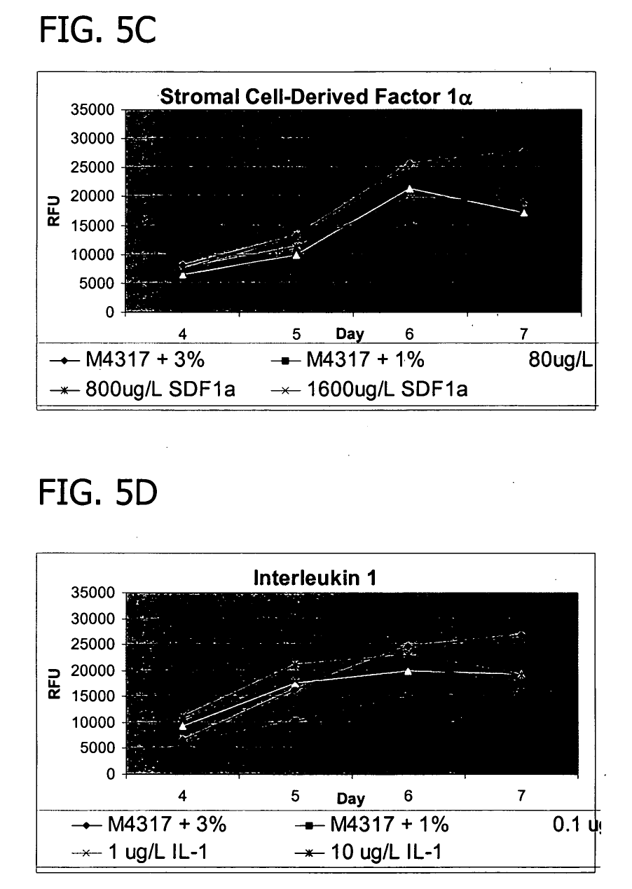

Using the approach used with respect to the HEK-293 cells, 17 positive receptors were generated, which are listed in Table 5. Of the initial 17 receptors gleaned from the microarray, 12 ligands were chosen for testing. These ligands were chosen in the same way as the ligands for the HEK-293 cells, based on signal versus controls as well as based on knowledge of the various factors. In this case, 4 of the chosen factors exhibited a positive effect on the proliferation of the WI-38 cells (FIG. 5). These 4 factors, basic fibroblast growth factor (bFGF), platelet-derived growth factor (PDGF), stromal cell-derived growth factor 1α (SDF1α), and interleukin-1 (IL-1), all gave significant increased proliferation, while the other 8 factors were either neutral or negative for proliferation.

TABLE 5

| Receptor | Ligand |
|---|---|
| Interferon gamma receptor 2 | IFNγ* |
| GDNF family receptor alpha 3 | Artemin* |
| interleukin-1 receptor-associated kinase 1 | IL-1* |
| chemokine (C-X-C motif), receptor 4 (fusin) | SDF1α* |
| transforming growth factor, beta receptor II (70-80 kD) | TGFβ* |
| interferon gamma receptor 1 | IFNγ* |
| platelet-derived growth factor receptor, beta polypeptide | PDGF AB* |
| fibroblast growth factor receptor 1 | bFGF* |
| AXL receptor tyrosine kinase | gas6 |
| insulin-like growth factor 1 receptor | IGF1* |
| interleukin 13 receptor, alpha 1 | IL-13* |
| bone morphogenetic protein receptor, type II | BMP-2* |
| fms-related tyrosine kinase 1 | VEGF* |
| fibroblast growth factor receptor 4 | aFGF |
| interleukin 2 receptor, gamma | IL-2 |
| interleukin 7 receptor | IL-7* |
| interleukin 2 receptor, alpha | IL-2 |

Using MegaCell™ MEM:F12 medium (Sigma-Aldrich Co., Product No. M4317), the FBS levels were reduced to 3% while maintaining good growth. Cells that were in log phase growth were used to harvest RNA for the microarray assays.

Signaling within a cell for increased growth can be stimulated in a variety of different ways. Combinations of the 4 chosen factors as listed above we added to cell culture medium to determine if the addition of more than 1 of the positive ligands would produce an increased amount of proliferation, or if the ligands would all stimulate the same downstream pathways, leading to no added benefit. This method allowed for the formulation of cell culture medium that it had not been possible to formulate prior to these assays.

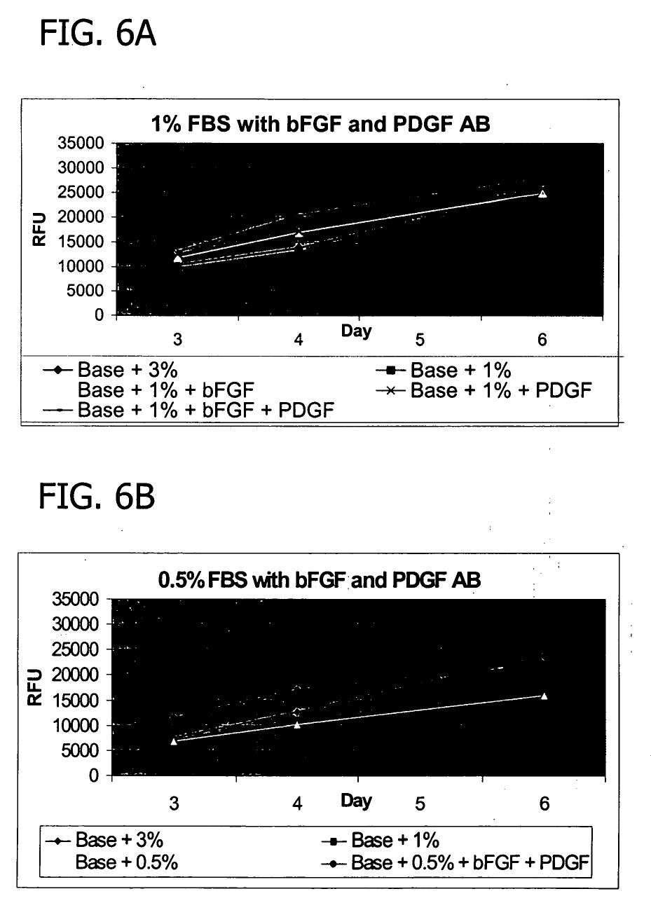

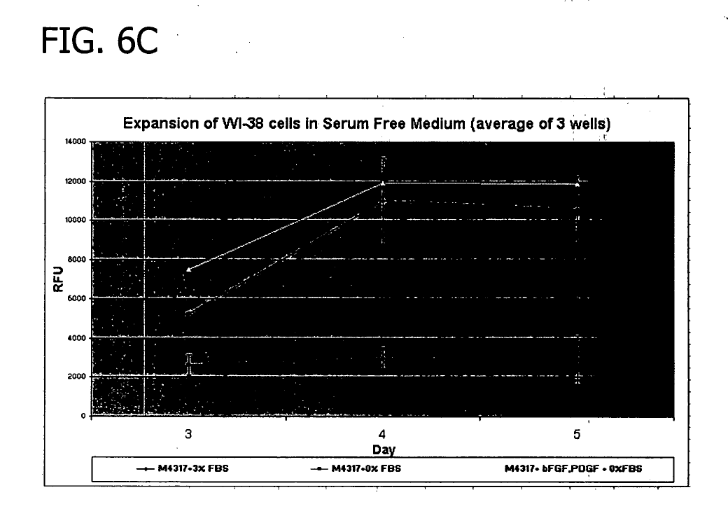

There was a positive interaction between bFGF and PDGF, which led to an increase in the proliferation of the cells when grown in 1% FBS (FIG. 6A), 0.5% FBS (FIG. 6B), and 0.0% FBS (i.e., serum-free medium) (FIG. 6C). The added benefit of the combination of growth factors allowed for the reduction in the FBS levels required for culture of the WI-38 cells without any loss in performance relative to 3% FBS. A medium was formulated accordingly of the following components.

| | Product | Final |
|---|---|---|
| Product | Number | Concentration |
| MegaCell ™ Minimum | M4317 | 1X |
| Essential Medium/Nutrient | | |
| Mixture F-12 Ham | | |
| L-Glutamine (200 mM) | G7513 | 4 mM |
| Fibroblast Growth Factor - | F0291 | 0.1 ug/ml |
| Basic human (bFGF) | | |
| Platelet-Derived Growth | P3326 | 0.01 ug/mL |
| Factor-AB human (PDGF) | | |

C. Chinese Hamster Ovary Cells

In order to further test the possibility of using the method as applied to HEK-293 cells to identify pathways in other cell lines, Chinese hamster ovary (CHO) cells were run on a microarray. Cells from the CHO-AP line produce alkaline phosphatase (AP). These cells are grown in a suspension culture in a serum-free CHO medium (Sigma-Aldrich Co., Product No. C5467).

Although the cDNA array from Takara is designed with human sequences, the array was probed using cDNA made from mRNA from the CHO cells. This attempt generated 22 positive receptors, of which 16 were tested (Table 6).

TABLE 6

| Receptor | Ligand |
|---|---|
| Interleukin 12 receptor, beta 2 | IL-12 |
| Colony stimulating factor 1 receptor | CSF1 |
| Burkitt lymphoma receptor 1 | BLC* |
| Chemokine receptor 9 | TECK* |
| Interleukin 11 receptor, alpha | IL-11 |
| Bone morphogenetic protein receptor, type IA | BMP2* |
| Fibroblast growth factor receptor 4 | aFGF |
| Interleukin 1 receptor-like 1 | IL-1* |
| Chemokine receptor 4 | MDC* |
| Chemokine receptor 1 | MCP3* |
| Platelet derived growth factor receptor, beta polypeptide | PDGF AB* |
| Transforming growth factor, beta receptor II | TGFβ* |
| Activin A receptor, type II | Activin A* |
| Macrophage stimulating 1 receptor | MSP* |
| Interferon gamma receptor 2 | IFNγ* |
| Bone morphogenetic protein receptor, type II | BMP2* |
| G protein-coupled receptor 9 | MIG* |
| Activin A receptor, type I | Activin A* |
| Fibroblast growth factor receptor 1 | bFGF* |
| Insulin-like growth factor 2 receptor | IGF2* |
| Autocrine motility factor receptor | PGI* |
| GDNF family receptor alpha 3 | Artemin* |

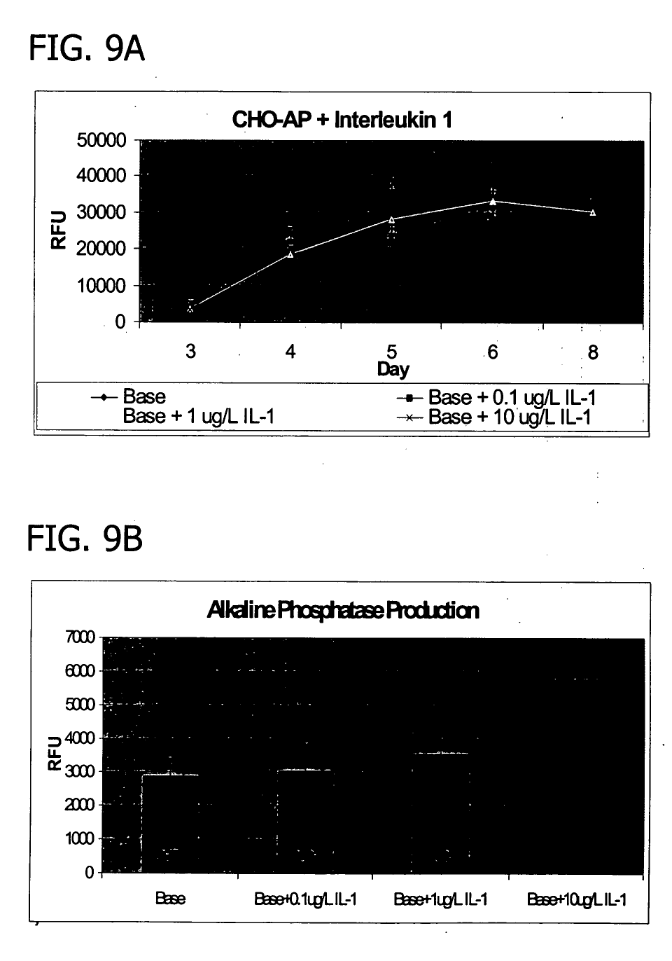

Of these 16 positive receptors, one in particular had a positive effect on the proliferation of the cells, interleukin-1 (FIG. 9A). Addition of interleukin-1 not only had an effect on the proliferation of the CHO-AP cells, but also increased the productivity of alkaline phosphatase (FIG. 9B). This technology will work for cells in different formats, for example, suspension versus attached, and from multiple species as demonstrated above.

### Example 2

### Membrane Array (Macroarray) and Protein/Antibody Array Analysis

Materials and Methods

In the following example, WI-38 cells were used. The cells are normal human fibroblast cells and are commercially available from ATCC(CCL-75).

The macroarray used herein was the SuperArray BioScience (Frederick, Md.), that was designed to determine the expression profile of a special group of genes, including matrix metalloproteinases (MMPs), integrins, proteases and protease inhibitors, all of which are involved in cell-cell and tissue-tissue interactions. cDNA made from mRNA from WI-38 cells was labeled and hybridized to the array.

RNA Isolation

mRNA isolation from WI-38 cells was isolated according to the RNA Isolation procedures of Example 1.

Preparation of Biotinylated WI-38 cDNA

WI-38 biotinylated cDNA was generated using mRNA isolated from WI-38 cells, anchored oligo dT Primer (04387), a nucleotide mix low in dTTP, Biol6-dUTP (Roche #1093070), RNase inhibitor (R2520), 5XMMLV Reverse transcriptase buffer (B0175), 0.1 M DTT, and M-MLV reverse transcriptase (M 1427). The RT reaction was performed at 42° C. for 2 hours. Subsequently, the mRNA was degraded with NaOH by incubating at 65° C. for 15 minutes. 1M Tris buffer was added to neutralize the reaction. Unincorporated dNTPs were removed by purification over a SigmaSpin size exclusion column (S5059). The biotinylated WI-38 cDNA was then stored at −20° C.

The biotinylation labeling efficiency of the WI-38 probe was tested by spotting dilutions of the probe onto a neutral nylon membrane (N3656). After spotting 1 μl of diluted probe onto the nylon membrane, it was UV crosslinked with 130 mJ/cm², blocked with blocking buffer (western blocking reagent, Roche, in maleic acid buffer), and rinsed with maleic acid buffer (0.1 M maleic acid (M0375), 0.1 M NaCl (S3014), pH 7.5 with NaOH(S5881)). The membrane was developed by incubation with streptavidin-peroxidase conjugate buffer (10% blocking buffer, 1 μg/ml streptavidin peroxidase (S2438) for 20 minutes, rinsed 3× in wash buffer III (maleic acid buffer with 0.3% Tween 20), rinsed 1×in maleic acid buffer, and drained. The membrane was then incubated with the peroxidase chemiluminescent substrate (CPS-1-60, Sigma-Aldrich Co.) according to its instructions. The membrane was then exposed to Kodak BioMax Light x-ray film for approximately 1 to 20 seconds (Z37,042-8, Sigma-Aldrich Co.) and developed using GBX developer and fixer (Z35,414-7, Sigma-Aldrich Co.).

Hybridization of WI-38 cDNA to Membrane Array (Macroarray)

A membrane array containing arrayed cDNA fragments from genes associated with extracellular matrix and adhesion molecules was used for this experiment (SuperArray, GEArray Q series, human extracellular matrix and adhesion molecules gene array, HS-010). Each cDNA fragment is printed in a tetra-spot format, which provides an easily identifiable pattern upon hybridization and development. All membrane transfers were performed using forceps. All hybridization and wash steps were performed in a 50 ml polypropylene conical tube with plug seal cap. The membrane was oriented in the tube such that the array side faced the inside of the tube and the solution. All detection steps were performed in a small plastic dish (i.e., box top from the pipet tips).

The protocol from the manufacturer was not followed; rather a procedure from the Sigma-Genosys, technical bulletin for the Panorama human cancer OligoArray (Sigma-Genosys, Produce No. G6667) was followed. The array was rinsed in 25 ml 2×SSPE buffer (Sigma-Aldrich Co., Product No. S2015). The membrane was then prehybridized in 5 ml prewarmed ArrayHyb Plus (Sigma-Aldrich Co., Product No. H7033) containing sonicated salmon testes DNA (Sigma-Aldrich Co., Product No. D7656), at 0.1 mg/ml for 30 minutes at 65° C. with gentle rotation. The entire WI-38 biotinylated cDNA probe was added to fresh prewarmed ArrayHyb Plus (no salmon testes DNA) and hybridized overnight at 65° C. with gentle rotation (approximately 22 hours). The signals were detected as previously described for testing the biotinylation cDNA probe spotted on a membrane.

Following hybridization, the membrane was transferred to 25 ml of blocking buffer and blocked for 2 hours at room temperature. The membrane was developed by incubation with streptavidin-peroxidase conjugate buffer (10% blocking buffer, 1 μg/ml streptavidin peroxidse, S2438) for 20 minutes. It was then rinsed with wash buffer III (maleic acid buffer with 0.3% Tween 20) 4×10 minutes each and then in maleic acid buffer for 5 minutes. The membrane was then incubated with the peroxidase chemiluminescent substrate (CPS-1-60, Sigma-Aldrich, Co.) according to its instructions. The membrane was then exposed to Kodak BioMax Light x-ray film for approximately 1 to 20 seconds (Z37,042-8, Sigma-Aldrich Co.) and developed using GBX developed and fixer (Z35,414-7, Sigma-Aldrich Co.).

Protein/Antibody Array

WI-38 protein containing cell extracts were tested for specific proteins using the Panorama Antibody Microarray Cell Signaling Kit (Sigma-Aldrich Co., Product No. CSAA-1). The labeling and detection procedure supplied with the antibody array was followed. Two T225 cell culture flasks containing adherent WI-38 cells were washed twice with 50 ml cold PBS, scraped using a cell scraper, and harvested/lysed directly in Buffer A (10 ml extraction/labeling buffer, 50 μl protease inhibitor cocktail (Sigma-Aldrich Co., Product No. P4495), 100 μl phosphatase inhibitor cocktail 1 (Sigma-Aldrich Co., Product No. P2850), 100 μl phophatase inhibitor cocktail II (Sigma-Aldrich Co., Product No. P5726), and 1.2 μl benzonase (5 units/μl)). The protein concentration was determined using the Bradford assay (Sigma-Aldrich Co., Product No. B6916) using a BSA standard (P0914) to prepare the standard curve. The protein concentration was approximately 1 mg/ml.

One ml (1 mg) of this WI-38 cell extract was added to a vial of Cy3 and a vial Cy5 dye (PA23001/PA25001, mono-Reactive NHS-ester dye Cy3/Cy5 sufficient for labeling 1 mg protein, Amersham). The dye solutions were then incubated for 30 minutes to 1 hour at room temperature to overnight at 4° C. Free dye was removed by size exclusion purification over 2 SigmaSpin columns (Sigma-Aldrich Co., Product No. S5059). The protein concentration of each sample following purification was again determined using a Bradford assay. The dye concentration was estimated using the absorbance maximum for each dye (A552 for Cy3 and A650 for Cy5) and the dye's extinction coefficients. The Dye to Protein (D/P) molar ratio was determined using 60 kDa as the average protein MW since these samples contain a mixture of cellular proteins.

This experiment was performed twice using two antibody array slides. In each case, the labeling efficiencies were low, <0.3, although the technical bulletin suggests using samples with >2 for best results. The entire SigmaSpin purified Cy3 and Cy5 samples were diluted in 5 ml Array Incubation buffer supplied with the kit and incubated for 45 minutes at room temperature, washed, dried and scanned using a Perkin-Elmer ScanArray Express at instrument settings (laser & PMT) selected to maximize signal while minimizing pixel saturation.

Results

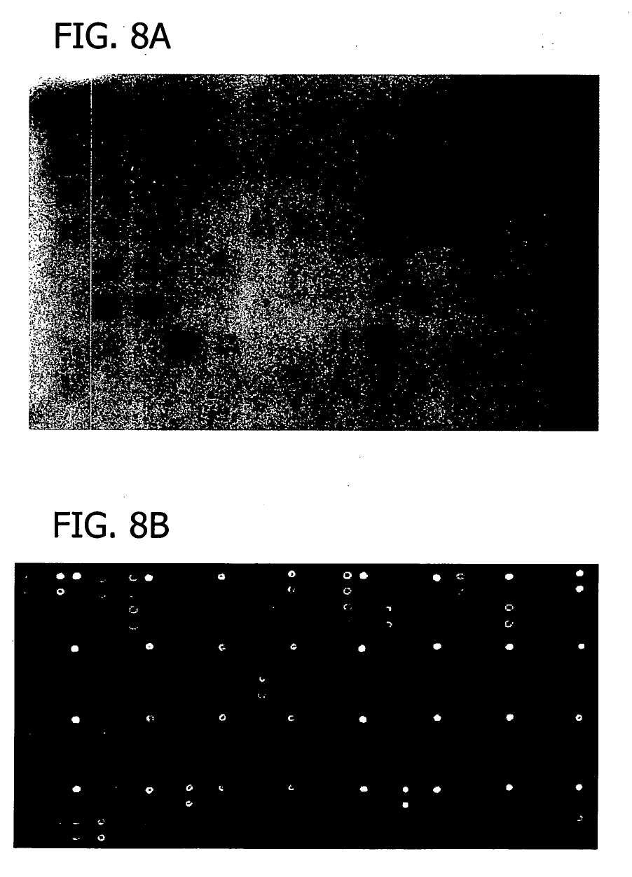

We were able to detect a wide variety of adhesion molecules present in the cells (FIG. 8A). It appeared that these fibroblasts would stick to almost any surface that they came in contact with, probably due to the abundance of adhesion molecules on their surface.

We also tested lysates from WI-38 to look at the levels of the specific proteins present in the cells. These lysates were tested on a Panorama Ab Microarray (Sigma-Aldrich Co.). FIG. 8B shows that there was a positive signal for several of the proteins represented on the array. We could easily use both of these technologies as alternatives to the cDNA microarray described previously.

### Example 3

### PCR Based Techniques

Materials and Methods

In the following example, Chinese hamster ovary (CHO) and WI-38 human fibroblast cells were used. These cells are further described above.

Generation of cDNA for PCR

Real-time fluorescent-based quantitative RT-PCR and standard gel-based RT-PCR was performed on WI-38 cells and CHO cells (both parental CHO-K1 and CHO-alkaline phosphatase expressing cells). cDNA was prepared using DNase treated total RNA isolated form WI-38 cells (as described previously in the microarray section). RNA and anchored oligo dT primer (Sigma-Aldrich Co., Product No. 04387) were incubated at 70° C. for 10 minutes and then chilled on ice. M-MLV RT buffer with DTT, M-MLV reverse transcriptase (Sigma-Aldrich Co., Product No. M1302), RNase inhibitor (Sigma-Aldrich Co., Product No. R2520), dNTP mix (Sigma-Aldrich Co., Product No. D7295) and water were added to the RNA with oligo dT primer. The reverse transcription reaction was incubated for 2 hours at 42° C. RNA was removed by treatment with NaOH and incubation at 70° C. for 15 minutes. 1 M Tris was added to neutralize the solution and it was then purified by size exclusion chromatography using a SigmaSpin column (Sigma-Aldrich Co., Product No. S5059).

Standard RT-PCR

The PCR reaction is assembled using cDNA (WI-38, CHO), 25 mM MgCl₂ (Sigma-Aldrich Co., Product No. M8787), water (W4502), forward and reverse primers, and JumpStart RedTaq ReadyMix (Sigma-Aldrich Co., Product No. P0982). Typically 40-200 ng template is used per reaction. The primer concentration is 1 micromolar each primer (forward and reverse). The JumpStart RedTaq ReadyMix is supplied as a 2× formulation and is diluted to 1× in the final reaction. Supplemental MgCl₂ is added at typically an additional 0.5 mM final concentration. Reactions were typically carried out in 96-ell PCR plates Sigma-Aldrich Co., Product No. Z37,490-3). Typical amplification conditions are 94° C. for 3 minutes followed by 35 cycles of 94° C. for 30 seconds, 57° C.-62° C. for 45 seconds to 1 minute, 70° C.-72° C. for 1 minute 30 seconds and a final extension step at 72° C. for 7 minutes. Following cycling, 5 μl samples were analyzed by horizontal agarose gel electrophoresis by loading directly into a 2.5% standard: wide range (3:1) agarose blend gel (A7431) in 1×TBE (T4415) running buffer. Bands were visualized by staining with ethidium bromide and images were captured using a BiORad Fluor-S imager.

Real-Time Quantitative RT-PCR

The PCR reaction is assembled using cDNA (WI-38, CHO), water (W4502), forward and reverse primers, and SYBR®Green JumpStart Taq Ready Mix (Sigma-Aldrich Co., Product No. S4438). Typically 20-200 ng template is used per reaction. The primer concentration is 1 micromolar each primer (forward and reverse). The SYBR® Green JumpStart Taq Ready Mix is supplied as a 2× formulation and is diluted to 1× in the final reaction. Reactions were carried out in 96-well PCR plates using the DNA Engine Opticon® 2 Continuous Fluorescence Detection System (MJ Research, Inc., Reno, Nev.) in a Hard-shell™ thin-well white well blue shell 96-well microplate (MJ Research, HSP-9635) sealed with ultra-clear flat optical strip caps (MJ Research, Reno, Nev.). Typical amplification conditions are 94° C. for 3 minutes followed by 40 cycles of 94° C. for 30 seconds, 60° C. for 30 seconds, 74° C. for 1 minute 30 seconds (plate read) followed by a melt curve analysis running from 50° C.-94° C. in increments of 0.2° C. with hold time of 1 second (plate read). The fluorescence value for each well is recorded during every cycle and represents the amount of product amplified to that point in the amplification reaction. The threshold cycle (Ct) is the point at which the flourescent signal becomes statistically significant above background and is determined using the Opticon 2 software. A higher concentration of template in the reaction will require a fewer number of cycles to reach its Ct value. The melt curve analysis performed for each sample was useful for product identification. Melt curve analysis can distinguish between the desired amplicon and primer dimer based on their differential melt curves. Following cycling, select samples (5 μl+1.5 μl 6× loading buffer (P7206) were analyzed by horizontal agarose gel electrophoresis on a 2.5% standard: wide range (3:1) agarose blend gel (A7431) in 1×TBE (T4415) running buffer. Bands were visualized by staining with ethidium bromide and images were captured using a BiORad Fluor-S imager.

Results

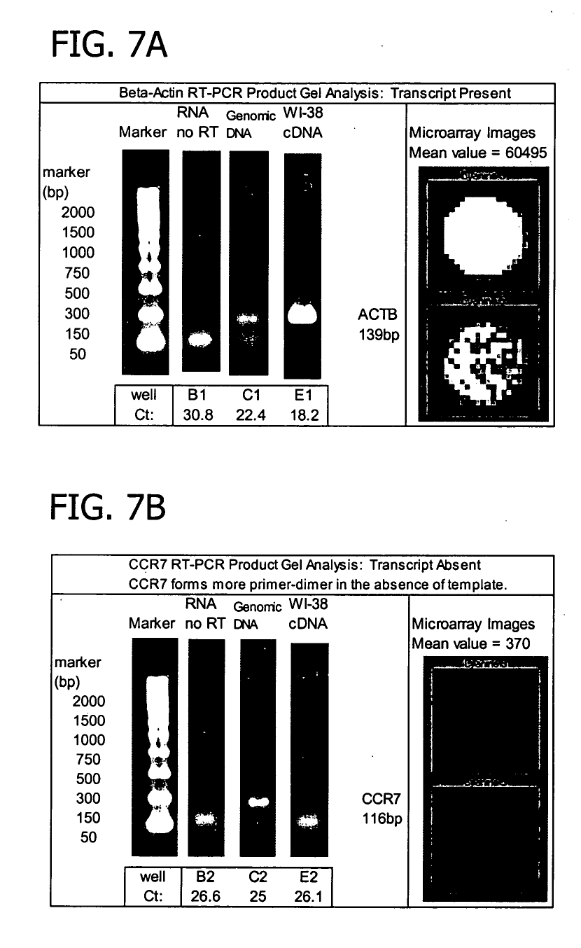

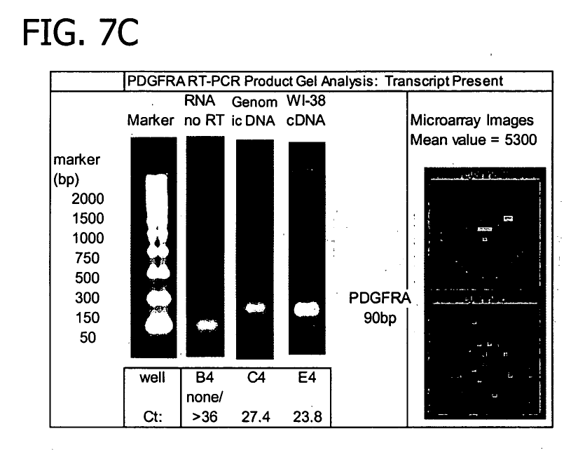

As discussed previously, microarray technology is a good way to detect the presence of mRNA for a given protein, but there are other ways to detect both the mRNA message as well as the protein itself. As an example of the various technologies, a receptor was selected which had shown itself as a positive on the microarray. FIG. 7 shows the positive signal from the PDGFR, stimulation of which had a positive effect on proliferation. It also shows corresponding data from beta-actin (housekeeping gene) as well as CCR7 (a receptor deemed negative on the microarray).

Primers were made to the mRNA for these proteins. The presence of the mRNA was detected using RT-PCR. The corresponding bands can also be seen in FIG. 7.

In order to get a quantifiable response to check for the amounts of message that were present within cell culture, primers were also created for use in quantitative PCR. FIG. 7 also shows a table with the CT values for the 3 components. All of these methods were used to identify the PDGF receptor as expressed in WI-38 cells.

### Example 4

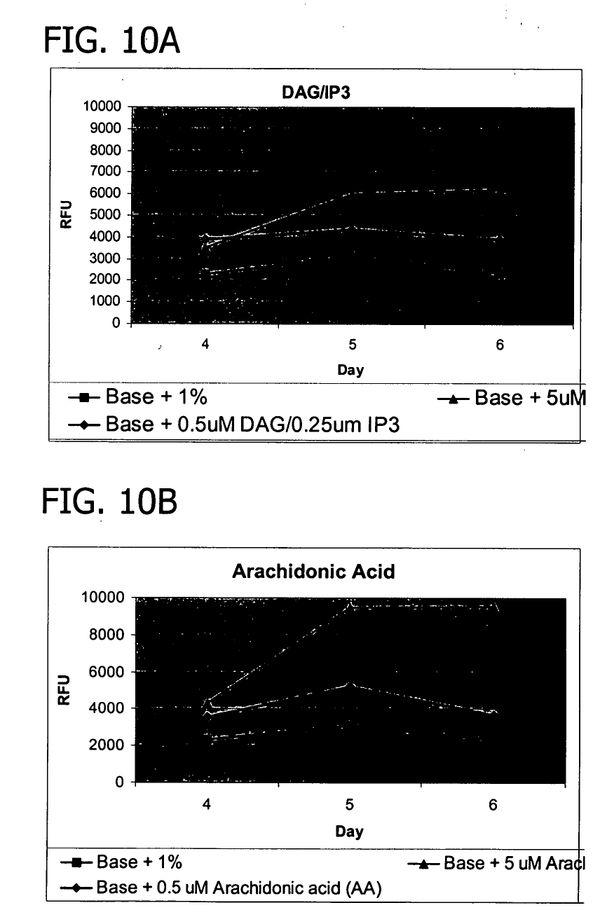

Due to the high cost of adding various growth factors and cytokines to a cell culture medium in sufficient quantities to have the desired impact, alternative methods to stimulate these pathways was sought (FIG. 10). Protein kinase C (PKC) is activated by several of the positive receptors listed in FIG. 10, and include EGF, bFGF, and SDF1α. The addition of arachidonic acid, which has been found to stimulate PKC in some systems, had a positive effect on proliferation (FIG. 10b). Endogenous intermediates such as IP3 and DAG (produced by the activation of phospholipase C) act via stimulation of PKC and release of intracellular Ca++. This combination also led to increased proliferation (FIG. 10A). Identifying growth factor/cytokine receptors can be used to identify the pertinent pathway, which can be effected in a variety of ways, not necessarily only the ligand for the receptor.

### Example 5

Various assays may be used to determine whether a molecule modulates a cellular activity. Such assays include the following.

Resazurin Assay

Cells were plated in each well of a 24-well tissue culture treated plate containing 1 ml of a base medium. The base medium contained the lowest amount of FBS required to maintain approximately half-maximal growth of the given cell type. Test conditions were performed in triplicate, with each test compound added to the base medium at three different concentrations. The cells were allowed to grow until they reached approximately 33% confluent. At this point, 100 μl of the resazurin solution from a resazurin based in vitro toxicology kit (Sigma-Aldrich Co., Product No. TOX-8) was added to each well. After sufficient incubation time to convert some of the resazurin, the fluorescence was measured on a HTS 7000 Plus BioAssay Reader (Perkin-Elmer, Boston, Mass.). Readings were taken once a day until the culture was confluent (typically about 4 days). A plate with base medium only (no cells) was used as a blank and subtracted from the RFU reading to establish the final RFU values.

Adhesion Assay

Cells were plated in the base medium at low density on various substrates using the BD BioCoat extracellular matrix coated plates (BD BioSciences, San Jose, Calif.). Starting 24 hours post plating, the cells were observed for both number of cells attached and morphology (i.e., degree of cell-spreading).

Alkaline Phosphatase Assay

On day 7 of the assay, the contents of each well were collected into 1.5 ml microfuge tubes. The cell suspensions were centrifuged at 16,000 rpm for five minutes. The supernatants were collected in 2 ml cryovials and the cell pellets were discarded. Each supernatant was diluted 1:10 and then assayed for the presence of Alkaline Phosphatase using the Alkaline Phosphatase Reporter Gene Assay Kit, Fluorescence (Product AP-F).

The invention being thus described, it will be obvious that the same may be varied in many ways. Such variations are not to be regarded as a departure from the spirit and scope of the invention, and all such modifications are intended to be included within the scope of the following claims.

What is claimed is:

1. A method for formulating a cell culture medium comprising:
detecting a sequence in a cell, the sequence being a nucleic acid sequence or an expressed amino acid sequence, and
formulating a cell culture medium to contain a molecule to modulate the detected sequence or its expression or to modulate a cellular process affected by the detected sequence or its expression.

2. A method for formulating a cell culture medium, said method comprising:
detecting a sequence in a cell, the sequence being a nucleic acid sequence or an expressed amino acid sequence;
determining whether a molecule modulates the sequence or its expression or modulates a cellular process affected by the sequence or its expression;
formulating a cell culture medium to contain a molecule to modulate the detected sequence or its expression or to modulate a cellular process affected by the detected sequence or its expression.

3. The method of claim 1, the method further comprising forming the formulated culture medium.

4. The method of claim 2, the method further comprising forming the formulated culture medium.

5. The method of claim 2, wherein the detected sequence is a mutant nucleic acid sequence.

6. The method of claim 2, wherein the detected sequence is RNA or a mutant RNA.

7. The method of claim 2, wherein the detected sequence is mRNA or mutant mRNA.

8. The method of claim 2, wherein the detected sequence is an expressed amino acid sequence.

9. The method of claim 2, wherein the sequence is detected using an array comprising a plurality of distinct biopolymers selected from the group consisting of antibodies, antibody fragments, polynucleotides, polynucleotide fragments, polypeptides, and polypeptide fragments.

10. The method of claim 9 wherein the distinct biopolymers are polynucleotides derived from a cell or complements to the polynucleotides derived from the cell.

11. The method of claim 2, wherein the detection of the nucleic acid sequence comprises isolating a nucleic acid sequence from a cell, amplifying the isolated nucleic acid sequence, and detecting the amplified nucleic acid sequence.

12. The method of claim 11 wherein the detected nucleic acid sequence encodes a polypeptide that affects a cellular process.

13. The method of claim 12 wherein the polypeptide is selected from the group consisting of intracellular receptors, cell-surface receptors, enzymes, growth factors, cytokines, interleukins, transcription factors, hormones, adhesion molecules, cadherins, and integrins.

14. The method of claim 11, wherein the nucleic acid sequence is a deoxyribozyme, a ribozyme, a micoRNA, or a nucleic acid analog.

15. The method of claim 2, wherein the sequence is an expressed amino acid sequence and the detection of this sequence comprises isolating the amino acid sequence with an antibody or fragment thereof.

16. The method of claim 15, wherein the antibody or antibody fragment specifically binds to a polypeptide selected from the group consisting of intracellular receptors, cell-surface receptors, enzymes, growth factors, cytokines, interleukins, transcription factors, hormones, adhesion molecules, cadherins, and integrins.

17. The method of claim 2, wherein the nucleic acid sequence is a deoxyribozyme, a ribozyme, a micoRNA, or a nucleic acid analog.

18. The method of claim 2, wherein the molecule is an expressed polypeptide affecting a cellular process selected from the group consisting of cell division, cell growth, cell proliferation, cell support, cell metabolism, and adhesion.

19. The method of claim 2, wherein said determination step comprises use of an assay selected from the group consisting of a cell growth or proliferation assay, an adhesion assay, a production assay, a post translational modification assay, a transfection assay, an apoptosis assay, a paracrine control assay, an RNA interfering assay, and an immortalization assay.

20. The method of claim 2 wherein the formulated culture medium contains less than 10% (v/v) of serum.

21. The method of claim 2, wherein the formulated culture medium is serum-free.

22. The method of claim 2, wherein the cell is derived from an organism selected from the group consisting of vertebrates, invertebrates, bacteria, plants, fungi and mammals.

23. The method of claim 22, wherein the bacteria are Archaebacteria or Eubacteria.

24. A formulated cell culture medium made by the method of claim 2.

25. A method for preparing a cell culture medium, said method comprising:
contacting a polynucleotide with an array comprising a plurality of biopolymers immobilized on the surface of the array, the polynucleotide being derived from a cell or the complement thereof;
detecting a bound pair formed between the polynucleotide and an immobilized biopolymer;
selecting a molecule for inclusion in a cell culture medium based on the members of the detected bound pair;
testing selected molecule to determine its effect on a cellular process of the cell; and
formulating a cell culture medium to include the selected molecule.

26. The method of claim 25, wherein the biopolymer is a nucleic acid and the bound pair is a hybridized nucleic acid pair.

27. A method for preparing a cell culture medium, said method comprising:
contacting a polypeptide with an array comprising a plurality of biopolymers immobilized on the surface of the array;
detecting a bound pair formed between the polypeptide and an immobilized biopolymer;
selecting a molecule for inclusion in a cell culture medium based on the members of the detected bound pair;
testing selected molecule to determine its effect on a cellular process of the cell; and; and
formulating a cell culture medium to include the selected molecule.

28. The method of claim 25, wherein the candidate component is added to a serum-free medium.

29. A cell culture medium made by the method of claims 25.

30. An array comprising at least one nucleic acid of, but less than the entire genome of, a Chinese hamster cell, said nucleic acid being immobilized on the surface of the array.

31. The array of claim 30, wherein the at least one nucleic acid is a plurality of nucleic acids.

32. The array of claim 30, wherein the at least one nucleic acid is a polynucleotide selected from the group consisting of Chinese hamster cell polynucleotides encoding or regulating the expression of receptors, enzymes, cytokines, interleukins, transcription factors, hormones, adhesion molecules, cadherins, and integrins.

33. The array of claim 30, wherein the polynucleotide is selected from the group consisting of a deoxyribozyme, a ribozyme, a microRNA, and a nucleic acid analog.

34. The array of claim 32, further comprising nucleic acids of Chinese hamster housekeeping genes.

35. A method for designing a cell culture medium adapted to support a cell line in a pre-defined manner, which method comprises:
(a) generating an expression profile of a cell line;
(b) identifying from the expression profile a set of biomolecules to evaluate for their effect on an end-point assay using the cell line;
(c) testing each biomolecule in the set for its effect in the endpoint assay, wherein each biomolecule that is determined to have a measurable effect in the end-point assay relative to a control, not containing the biomolecule, is considered a positive biomolecule; and
(d) formulating a cell culture medium for the cell line by adding a positive biomolecule to a basal medium to form a modified medium, and determining whether the modified medium is sufficient to support the cell line in the pre-defined manner.

36. The method according to claim 35 further comprising repeating step (d) with additional positive biomolecules until the modified medium is able to support the cell line in the predefined manner.

37. A method according to claim 35 wherein the expression profile is generated on an array comprising a plurality of distinct biopolymers immobilized thereon.

38. A method according to claim 37 wherein the array is a microarray or a macroarray.

39. A method according to claim 37 wherein the distinct biopolymers are polynucleotide sequences that partially encode, or are complementary to polynucleotide sequences that partially encode, polypeptides of known function.

40. A method according to claim 39 wherein a plurality of polynucleotide probes derived from the cell line are contacted with the biopolymers immobilized on the array under hybridizing conditions.

41. A method according to claim 40 further comprising detecting those probes that hybridize to the biopolymers immobilized on the array.

42. A method according to claim 41 wherein the hybridization of each probe to a distinct biopolymer indicates that the cell line expresses a polypeptide partially encoded by the biopolymer.

43. A method according to claim 37 wherein the distinct biopolymers are selected from the group consisting of antibodies, antibody fragments, polypeptide fragments and combinations thereof.

44. A method according to claim 43 wherein a polypeptide sample derived from the cell line is contacted with the array, and those polypeptides that specifically bind to an antibody or antibody fragment are detected and provide the expression profile.

45. A method according to claim 35 wherein the expression profile is generated by:
(a) making primer sets for a plurality of biopolymers;
(b) using the primer sets to amplify polynucleotide sequences isolated from the cell line; and
(c) detecting each amplified sequence, wherein the amplification of a particular sequence confirms that the cell line expresses a polypeptide encoded by the amplified sequence.

46. A method according to claim 45 wherein the polynucleotide sequences isolated from the cell line are reverse transcribed prior to amplification with the primer sets.

47. A method according to claim 45 wherein the primer sets are designed based on polynucleotide sequences that encode polypeptides that participate in biologically significant cellular processes.

48. A method according to claim 47 wherein the polypeptide is selected from the group consisting of intracellular receptors, cell-surface receptors, enzymes, growth factors, cytokines, interleukins, transcription factors, hormones, adhesion molecules, cadherins, integrins, deoxyribozymes and ribozymes.

49. A method according to claim 35 wherein the expression profile is generated by:
(a) preparing a protein sample from the cell line and separating the sample in two dimensions; and
(b) using a plurality of antibodies to identify specific proteins that are expressed by the cell line, wherein the binding of an antibody to a protein confirms that the cell line expresses the protein recognized by the antibody.

50. A method according to claim 49 wherein the antibody or antibody fragment specifically binds to a polypeptide selected from the group consisting of intracellular receptors, cell-surface receptors, enzymes, growth factors, cytokines, interleukins, transcription factors, hormones, adhesion molecules, cadherins, and integrins.

51. A method according to claim 37 wherein the biopolymer encodes a portion of a polypeptide, is an antibody or antibody fragment that specifically binds to a polypeptide or is itself a fragment of a polypeptide that is selected from the group consisting of intracellular receptors, cell-surface receptors, enzymes, growth factors, cytokines, interleukins, transcription factors, hormones, adhesion molecules, cadherins, integrins, deoxyribozymes and ribozymes.

52. A method according to claim 35 wherein the biomolecule is a polypeptide in a cellular process selected from the group consisting of cell division, cell growth, cell metabolism and adhesion.

53. A method according to claim 35 wherein the biomolecule is a small molecule.

54. A method according to claim 35 wherein the endpoint assay is selected from the group consisting of a cell growth or proliferation assay, an adhesion assay, a production assay, a post translational modification assay, a transfection assay, an apoptosis assay, a paracrine control assay and an immortalization assay.

55. A method according to claim 35 wherein the modified medium that supports the cell line is a low serum medium.

56. A method according to claim 55 wherein the medium contains less than 10%(wt) serum.

57. A method according to claim 56 wherein the medium contains less than 7.5%(wt) serum.

58. A method according to claim 57 wherein the medium contains less than 5%(wt) serum.

59. A method according to claim 58 wherein the medium contains between 1%-3%(wt) serum.

60. A method according to claim 59 wherein the medium contains less than 1%(wt) serum.

61. A method according to claim 60 wherein the medium is serum-free.

62. A method according to claim 35 wherein the cell line is derived from an organism selected from the group consisting of vertebrates, invertebrates, bacteria, plants, fungi and mammals.

63. A method according to claim 62 wherein the cell line is derived from a mammal.

64. A method according to claim 35 wherein the cell line is an immortalized cell line or a primary cell line.

65. A method according to claim 35 wherein the cell line is adapted to grow in suspension or is adapted to grow when attached to a substrate surface.

66. A cell culture medium made by the process of claims 35, 37, 43, 44, 45 or 46.

67. A method for identifying biomolecules for use in designing a cell culture medium adapted to support a cell line in a pre-defined manner comprising:
(a) generating a pool of polynucleotide probes that are complementary to polynucleotide sequences that encode fragments of polypeptides expressed by a cell line;
(b) contacting the pool of polynucleotide probes under hybridizing conditions with at least one array comprising a plurality of biopolymers immobilized on the surface of the array, each biopolymer encoding a fragment of a distinct polypeptide that participates in a biologically significant cellular process;
(c) generating an expression profile by detecting each polynucleotide probe that hybridizes to each biopolymer; and
(d) selecting biomolecules, based on the expression profile, to be candidate components for a cell culture medium based on the function of the polypeptide partially encoded by the biopolymers to which a probe hybridizes.

68. A method for preparing a serum-free cell culture medium that is sufficient to support a cell line, which method comprises:
(a) generating a pool of polynucleotide probes that are complementary to polynucleotide sequences that encode fragments of polypeptides expressed by a cell line;
(b) contacting the pool of polynucleotide probes under hybridizing conditions with at least one array comprising a plurality of biopolymers immobilized on the surface of the array, each biopolymer encoding a fragment of a distinct polypeptide that participates in a biologically significant cellular process;
(c) generating an expression profile by detecting each polynucleotide probe that hybridizes to each biopolymer;
(d) selecting biomolecules, based on the expression profile, to be candidate components for a serum-free medium based on the function of the polypeptide partially encoded by the biopolymers to which a probe hybridizes;
(e) testing each candidate component to determine its effect on growth and/or proliferation of the cell line, and designating those candidate components that increase growth and/or proliferation of the cell line as positive biomolecules; and
(f) adding a positive biomolecule to a serum-free basal medium and evaluating the growth and/or proliferation of the cell line in the modified medium.

69. A process according to claim 66 further comprising repeating step (f) with additional positive biomolecules until the modified medium is able to support the cell line.

70. A cell culture medium made by the method of claims 67, 68 or 69.

71. An array for designing and/or optimizing cell culture medium, which comprises a pool of biopolymers immobilized on a surface of a substrate, each biopolymer selected from the group consisting of polynucleotides encoding fragments of distinct polypeptides that are participants in biologically significant cellular processes, antibodies or antibody fragments that specifically bind to polypeptides that are participants in biologically significant cellular processes and fragments of polypeptides that are participants in biologically significant cellular processes.

72. An array according to claim 71 wherein each biopolymer is a distinct polynucleotide and encodes a portion of a polypeptide selected from the group consisting of intracellular receptors, cell-surface receptors, enzymes, growth factors, cytokines, interleukins, transcription factors, hormones, adhesion proteins, cadherins, and integrins.

73. An array according to claim 72 wherein the polypeptide is a participant in a pathway selected from the group consisting of cell division, cell growth, cell metabolism and adhesion.

74. An array according to claim 71 wherein each biopolymer is a distinct antibody or antibody fragment that specifically binds to an epitope on a polypeptide selected from the group consisting of intracellular receptors, cell-surface receptors, enzymes, growth factors, cytokines, interleukins, transcription factors, hormones, adhesion proteins, cadherins, and integrins.

75. An array according to claim 74 wherein the polypeptide is a participant in a pathway selected from the group consisting of cell division, cell growth, cell metabolism and adhesion.

76. A method for designing or optimizing a cell culture medium comprising using the array of claim 71 to generate an expression profile from which candidate biomolecules are identified for further testing as components in the cell culture medium.

77. A cell culture medium made using the microarray according to claim 71.
